(19) United States
(12) Patent Application Publication
(10) Pub. No.: US 2024/0100021 A1
(43) Pub. Date: Mar. 28, 2024

(54) Combination Therapy Schedules to Treat Cancer

(71) Applicant: BOARD OF REGENTS, THE UNIVERSITY OF TEXAS SYSTEM, Austin, TX (US)

(72) Inventors: Borje S. Andersson, Houston, TX (US); Benigno C. Valdez, Missouri City, TX (US)

(73) Assignee: BOARD OF REGENTS, THE UNIVERSITY OF TEXAS SYSTEM, Austin, TX (US)

(21) Appl. No.: 18/262,176

(22) PCT Filed: Jan. 19, 2022
(86) PCT No.: PCT/US2022/012989

§ 371 Date: Jul. 19, 2023

Related U.S. Application Data
(60) Provisional application No. 63/139,683, filed on Jan. 20, 2021.

(51) Int. Cl. A61K 31/4178; A61K 31/635; A61K 31/7068; A61K 31/7076; A61P 35/02

(52) U.S. Cl. CPC A61K 31/4178; A61K 31/635; A61K 31/7068; A61K 31/7076; A61P 35/02

(57) ABSTRACT

Aspects of the present disclosure are directed to methods for treating a subject having cancer. Certain aspects relate to treatment with an anthracene derivative after treatment with one or more pyrimidine analog antimetabolites. Further aspects relate to methods for improving the efficacy of one or more pyrimidine analog antimetabolites by administering to a subject a therapeutically effective amount of an anthracene derivative after administration of the one or more pyrimidine analog antimetabolites.

This application claims benefit of priority of U.S. Provisional Application No. 63/139,683, filed Jan. 20, 2021, which is hereby incorporated by reference in its entirety.

### BACKGROUND

### I. Field of the Disclosure

Aspects of this disclosure relate, generally, to at least the fields of cancer biology and medicine and, more specifically, to methods to improve the therapeutic benefit of bisantrene and analogs and derivatives thereof, particularly in the treatment of cancer.

### II. Background

While many advances have been made from basic scientific research to improvements in practical patient management, there still remains tremendous frustration in the rational and successful discovery of useful therapies for life-threatening diseases such as cancer. The complexity of the genetic and epigenetic abnormalities of cancer underscores the importance of finding drugs that efficiently control the proliferation of cancer cells and counteract the inherent and acquired drug resistance of the cancer cells. To achieve increased cytotoxic efficacy in treating cancer, drugs with different mechanisms of action have been combined, but overall survival remains poor. Unfortunately many of cytotoxic compounds that successfully meet preclinical testing and federal regulatory requirements for clinical evaluation are either unsuccessful or disappointing in human clinical trials, and a number of compounds that reach commercialization have limited clinical utility due to their poor efficacy as monotherapies (<25% response rates) and untoward dose-limiting side-effects (e.g., myelosuppression, neurotoxicity, cardiotoxicity, gastrointestinal toxicities, or other significant side effects).

Maximizing cytotoxic efficacy and reducing unwanted side effects are most beneficial to a patient's treatment outcome. Therefore, there exists a need in the art for identification of more efficacious drugs and their use in effective combination strategies with other available cytotoxic agents to treat cancer.

### SUMMARY

Aspects of the present disclosure address needs in the art by providing methods for treating subjects with cancer (e.g., acute leukemias of childhood) and methods for improving the efficacy of different classes of cytotoxic agents by administering the different classes of cytotoxic agents in a particular sequence. Accordingly, provided herein, in some aspects, are methods for treating a subject with cancer comprising (a) administering to the subject a therapeutically effective amount of one or more first cancer therapies or cytotoxic agents; and (b), subsequent to (a), administering to the subject a therapeutically effective amount of one or more second cancer therapies or cytotoxic agents. In some embodiments, the one or more first cancer therapies or cytotoxic agents comprise one or more pyrimidine analog antimetabolites. In some embodiments, the one or more second cancer therapies or cytotoxic agents comprise an anthracene derivative.

Embodiments of the disclosure include methods for treating a subject having cancer, methods for improving the efficacy of cytotoxic agents used to treat a subject having cancer, methods for sensitizing a subject with cancer to one or more cytotoxic agents, methods for identifying a subject with cancer as a candidate for a combination therapy, and methods and compositions for treating a subject having an acute leukemia of childhood. Methods of the disclosure can include 1, 2, 3, 4, 5, 6, or more of the following steps: providing a first cytotoxic agent to a subject, providing one or more pyrimidine analog antimetabolites to a subject, providing a second cytotoxic agent to a subject, providing an anthracene derivative to a subject, providing a combination therapy to a subject, providing an alternative therapy to a subject, determining a subject to have cancer, providing two or more types of cancer therapy to a subject, identifying one or more cytotoxic agents as being in need of improved efficacy, and identifying a subject as being a candidate for a combination therapy comprising one or more pyrimidine analog antimetabolites and an anthracene derivative. Certain embodiments of the disclosure may exclude one or more of the preceding elements and/or steps.

Disclosed herein, in some aspects, is a method for treating a subject for cancer, the method comprising (a) administering to the subject a therapeutically effective amount of one or more pyrimidine analog antimetabolites; and (b), subsequent to (a), administering to the subject a therapeutically effective amount of an anthracene derivative. In some embodiments, administering the anthracene derivative subsequent to administration of the one or more pyrimidine analog antimetabolites results in a significant, synergistic, cytotoxic effect.

In some embodiments, the anthracene derivative is bisantrene or a derivative or analog thereof. In some embodiments, the anthracene derivative is administered at a dose of between 0.05 mg/m² to 5000 mg/m². In some embodiments, the anthracene derivative is administered at a dose of between 0.1 mg/m² to 2500 mg/m². In some embodiments, the anthracene derivative is administered at a dose of between 1 mg/m² to 1000 mg/m². In some embodiments, the anthracene derivative is administered at a dose of between 50 mg/m² to 500 mg/m².

In some embodiments, the one or more pyrimidine analog antimetabolites comprise two or more pyrimidine antimetabolites. In some embodiments, the one or more pyrimidine analog antimetabolites comprise cytarabine, fludarabine, cladribine, clofarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, 2′-C-cyano-2′-deoxy-arabinofuranosylcytosine, or a combination thereof. In some embodiments, the one or more pyrimidine analog antimetabolites comprise cytarabine, fludarabine, cladribine, clofarabine, or a combination thereof. In some embodiments, the one or more pyrimidine analog antimetabolites comprise two or more of cytarabine, fludarabine, cladribine, and clofarabine. In some embodiments, the one or more pyrimidine analog antimetabolites comprise fludarabine and clofarabine.

In some embodiments, the one or more one or more pyrimidine analog antimetabolites comprise cytarabine, and the cytarabine is administered at a dose of between 1 mg/m² and 1000 mg/m². In some embodiments, the cytarabine is administered at a dose of between 5 mg/m² and 500 mg/m². In some embodiments, the cytarabine is administered at a dose of between 25 mg/m² and 250 mg/m². In some embodiments, the cytarabine is administered at a dose of between 50 mg/m² and 150 mg/m².

In some embodiments, the one or more one or more pyrimidine analog antimetabolites comprise fludarabine, and wherein the fludarabine is administered at a dose of between 0.25 mg/m² and 250 mg/m². In some embodiments, the fludarabine is administered at a dose of between 1.25 mg/m² and 125 mg/m². In some embodiments, the fludarabine is administered at a dose of between 2.5 mg/m² and 60 mg/m². In some embodiments, the fludarabine is administered at a dose of between 10 mg/m² and 40 mg/m².

In some embodiments, the one or more one or more pyrimidine analog antimetabolites comprise cladribine, and wherein the cladribine is administered at a dose of between 0.001 mg/kg and 1 mg/kg. In some embodiments, the cladribine is administered at a dose of between 0.005 mg/kg and 0.5 mg/kg. In some embodiments, the cladribine is administered at a dose of between 0.01 mg/kg and 0.25 mg/kg. In some embodiments, the cladribine is administered at a dose of between 0.05 mg/kg and 0.2 mg/kg.

In some embodiments, the one or more one or more pyrimidine analog antimetabolites comprise clofarabine, and wherein the clofarabine is administered at a dose of between 0.5 mg/m² and 500 mg/m². In some embodiments, the clofarabine is administered at a dose of between 1 mg/m² and 250 mg/m². In some embodiments, the clofarabine is administered at a dose of between 5 mg/m² and 100 mg/m². In some embodiments, the clofarabine is administered at a dose of between 25 mg/m² and 75 mg/m².

In some embodiments, the anthracene derivative is administered within 1 week, within 2 weeks, within 3 weeks, or within 1 month after administration of the one or more pyrimidine analog antimetabolites. In some embodiments, the anthracene derivative is administered within 1 week after administration of the one or more pyrimidine analog antimetabolites. In some embodiments, the anthracene derivative is administered within 1 day, within 2 days, within 3 days, within 4 days, within 5 days, or within 6 days after administration of the one or more pyrimidine analog antimetabolites. In some embodiments, the anthracene derivative is administered within 1 day after administration of the one or more pyrimidine analog antimetabolites. In some embodiments, the anthracene derivative is administered within 23 hours, within 22 hours, within 21 hours, within 20 hours, within 19 hours, within 18 hours, within 17 hours, within 16 hours, within 15 hours, within 14 hours, within 13 hours, within 12 hours, within 11 hours, within 10 hours, within 9 hours, within 8 hours, within 7 hours, within 6 hours, within 5 hours, within 4 hours, within 3 hours, within 2 hours, or within 1 hour after administration of the one or more pyrimidine analog antimetabolites. In some embodiments, the anthracene derivative is administered within 12 hours after administration of the one or more pyrimidine analog antimetabolites.

In some embodiments, multiple doses of the one or more pyrimidine analog antimetabolites are administered. In some embodiments, the method comprises administering multiple doses of the one or more pyrimidine analog antimetabolites, and the multiple doses are administered on 1, 2, 3, 4, 5, 6, 7, 8, 9, 10, 11, 12, 13, 14, 15, 16, 17, 18, 19, 20, 21, 22, 23, 24, 25, 26, 27, or 28 consecutive days. In some embodiments, the method comprises administering multiple doses of the one or more pyrimidine analog antimetabolites, and the multiple doses are administered on 1, 2, 3, 4, 5, 6, 7, 8, 9, 10, 11, 12, 13, 14, 15, 16, 17, 18, 19, 20, 21, 22, 23, 24, 25, 26, 27, or 28 non-consecutive days. In some embodiments, the anthracene derivative is administered after administration of every dose of the multiple doses of the one or more pyrimidine analog antimetabolites. In some embodiments, the anthracene derivative is administered between doses of the multiple doses of the one or more pyrimidine analog antimetabolites.

In some embodiments, the anthracene derivative or the one or more pyrimidine analog antimetabolites are administered intratumorally, intravenously, intramuscularly, intraperitoneally, subcutaneously, intraarticularly, intrasynovially, intrathecally, orally, topically, through inhalation, or through a combination of two or more routes of administration. In some embodiments, the anthracene derivative and the one or more pyrimidine analog antimetabolites are administered via the same route of administration. In some embodiments, the anthracene derivative and the one or more pyrimidine analog antimetabolites are administered via different routes of administration.

In some aspects, the method further comprises administering to the subject a BH3 mimetic. In some embodiments, the BH3 mimetic is ABT-199 (venetoclax), ABT-737, ABT-263 (navitoclax), WEHI-539, BXI-61, BXI-72, GX15-070 (obatoclax), S1, JY-1-106, apogossypolone, BI97C1 (sabutoclax), TW-37, MIM1, MS1, BH3I-1, UMI-77, or marinopyrrole A (maritoclax). In some embodiments, the BH3 mimetic is ABT-199 (venetoclax), ABT-737, or ABT-263 (navitoclax). In some embodiments, the BH3 mimetic is ABT-199. In some embodiments, the BH3 mimetic is administered at a dose of between 1 mg/kg and 1000 mg/kg. In some embodiments, the BH3 mimetic is administered at a dose of between 5 mg/kg and 500 mg/kg. In some embodiments, the BH3 mimetic is administered at a dose of between 25 mg/kg and 250 mg/kg. In some embodiments, the BH3 mimetic is administered at a dose of between 50 mg/kg and 150 mg/kg.

In some embodiments, the cancer is breast cancer, ovarian cancer, renal cancer, small-cell lung cancer, non-small cell lung cancer, Hodgkin's lymphoma, non-Hodgkin's lymphoma, acute myelocytic leukemia, acute lymphocytic leukemia, melanoma, gastric cancer, adrenal cancer, head and neck cancer, hepatocellular cancer, hypernephroma, bladder cancer, acute leukemias of childhood, chronic lymphocytic leukemia, prostate cancer, glioblastoma, and myeloma. In some embodiments, the cancer is an acute leukemia of childhood. In some embodiments, the cancer is acute myelocytic leukemia.

In some embodiments, the cancer is colon cancer, prostate cancer, lung cancer, melanoma, or breast cancer. In some embodiments, the cancer is lung cancer. In some embodiments, the lung cancer is non-small cell lung cancer. In some embodiments, the cancer is metastatic cancer.

“Individual, “subject,” and “patient” are used interchangeably and can refer to a human or non-human.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The results of treatment can be determined by methods known in the art, such as determination of reduction of pain as measured by reduction of requirement for administration of opiates or other pain medication, determination of reduction of tumor burden, determination of restoration of function, or other methods known in the art.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristics of the disclosure.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1B show the cytotoxicity of bisantrene (Bis) and ABT199 in combination with various nucleoside analogs. OCI-AML3 cells (FIG. 1A) and MOLM14 AML cells (FIG. 1B) were exposed to the indicated concentrations of Bis, cytarabine (Ara-C), cladribine (Clad), fludarabine (Flu), clofarabine (Clo) and ABT199, alone or in combination, for 48 hrs prior to determination of relative proliferation by MTT assay and apoptosis by Annexin V (Ann V) assay. The asterisk (*) indicates statistically significant difference (P<0.05) between drug combination with and without ABT199. The results are averages of at least three independent experiments. To determine drug synergism, cells were exposed to various drug combinations at constant ratio concentrations for 48 hrs prior to MTT assay. The relationships between the calculated combination indexes (CI) and fraction affected (Fa) are shown below the bar graphs. CI<1.0 indicates synergism. The graphs are representatives of two independent experiments.

FIGS. 2A-2D demonstrate the effects of Bis, nucleoside analogs and ABT199 on the status of molecular markers of apoptosis. Cells were exposed to the indicated drugs for 48 hrs, harvested, and analyzed by Western blotting (FIG. 2A and FIG. 2B), caspase 3 enzymatic assay (FIG. 2C), and DNA agarose gel electrophoresis (FIG. 2D). Other legends and abbreviations are similar to FIGS. 1A and 1B.

FIGS. 3A-3D demonstrate drug-mediated changes in the production of reactive oxygen species (ROS) and mitochondrial membrane potential (MMP). OCI-AML3 and MOLM14 cells were exposed to indicated drug(s) for 48 hrs prior to flow cytometric analysis to determine ROS production (FIG. 3A and FIG. 3B) and status of MMP (FIG. 3C and FIG. 3D) as described. Asterisk (*) indicates statistically significant difference (P<0.05) between drug combinations with and without ABT199. The results are expressed as the mean±standard deviation of at least three independent experiments.

FIGS. 4A-4B demonstrate the effects of drug sequence on cytotoxicity. Cells were exposed to the first drug(s) for 24 hrs, then the second drug(s) was/were added for another 24 hrs prior to analysis using MTT (FIG. 4A) and Annexin V (FIG. 4B) assays. The asterisk (*) indicates statistically significant difference (P<0.05). The results are expressed as the mean±standard deviation of five independent experiments.

### DETAILED DESCRIPTION

The present disclosure is based, at least in part, on the surprising discovery that when the anthracene xenobiotic bisantrene is combined with agents from a different class of cytotoxic agents, for example, one or more pyrimidine analog antimetabolites, which have different modes of action and affect different metabolic pathways, the sequencing of the bisantrene and the one or more pyrimidine analog antimetabolites trigger varying effects. Notably, when the one or more pyrimidine analog antimetabolites are followed by bisantrene, a significant, synergistic, cytotoxic effect is observed, but when the bisantrene precedes the one or more pyrimidine analog antimetabolites, the effects are at best additive and at worst antagonistic, thereby potentiating risk of treatment failure and preventing eradication of cancer.

Accordingly, in some embodiments, disclosed are methods for treating a subject for cancer comprising (a) administering to the subject a therapeutically effective amount of one or more cancer therapies; and (b), subsequent to (a), administering to the subject a therapeutically effective amount of an anthracene derivative. In some embodiments, the one or more cancer therapies comprise one or more pyrimidine analog antimetabolites. Thus, in some embodiments, the method comprises (a) administering to the subject a therapeutically effective amount of one or more pyrimidine analog antimetabolites; and (b), subsequent to (a), administering to the subject a therapeutically effective amount of an anthracene derivative.

### I. Therapeutic Methods

Aspects of the disclosure are directed to compositions and methods of administering therapeutically effective amounts of one or more cancer therapies to a subject or patient in need thereof. In some embodiments, the one or more cancer therapies comprise a cytotoxic agent. In some embodiments, the one or more cancer therapies comprise an anthracene derivative, which may be bisantrene. In some embodiments, the one or more cancer therapies comprise one or more pyrimidine analog antimetabolites, which may comprise cytarabine, fludarabine, cladribine, clofarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, 2′-C-cyano-2′-deoxy-arabinofuranosylcytosine, or a combination thereof. Administration of one or more pyrimidine analog antimetabolites may comprise administration of at least 1, 2, 3, 4, 5, or more pyrimidine analog antimetabolites. In some embodiments, the one or more pyrimidine analog antimetabolites comprise two or more of cytarabine, fludarabine, cladribine, and clofarabine. In some embodiments, the one or more pyrimidine analog antimetabolites comprise fludarabine and clofarabine. In some embodiments, the one or more cancer therapies comprise a BH3 mimetic, which may be ABT-199 (venetoclax), ABT-737, ABT-263 (navitoclax), WEHI-539, BXI-61, BXI-72, GX15-070 (obatoclax), S1, JY-1-106, apogossypolone, BI97C1 (sabutoclax), TW-37, MIM1, MS1, BH3I-1, UMI-77, or marinopyrrole A (maritoclax). Any of these cancer therapies may also be excluded. Combinations of these therapies may also be administered.

The compositions of the disclosure may be used for in vivo, in vitro, or ex vivo administration. The route of administration of the composition may be, for example, intratumoral, intravenous, intramuscular, intraperitoneal, subcutaneous, intraarticular, intrasynovial, intrathecal, oral, topical, through inhalation, or through a combination of two or more routes of administration. The cancer therapies may be administered via the same or different routes of administration.

The term “cancer,” as used herein, may be used to describe a solid tumor, metastatic cancer, or non-metastatic cancer. In certain embodiments, the cancer may originate in the blood, bladder, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, disclosed are methods for treating cancers comprising breast cancer, ovarian cancer, renal cancer, small-cell lung cancer, non-small cell lung cancer, Hodgkin's lymphoma, non-Hodgkin's lymphoma, acute myelocytic leukemia, acute lymphocytic leukemia, melanoma, gastric cancer, adrenal cancer, head and neck cancer, hepatocellular cancer, hypernephroma, bladder cancer, acute leukemias of childhood, chronic lymphocytic leukemia, prostate cancer, glioblastoma, and myeloma. In some embodiments, the cancer is an acute leukemia of childhood. In some embodiments, the cancer is acute myelocytic leukemia. In some embodiments, the cancer is lymphoma. In some embodiments, the cancer is breast cancer. In some embodiments, the cancer is ovarian cancer.

In some embodiments, the cancer therapy comprises a local cancer therapy. In some embodiments, the cancer therapy comprises a systemic cancer therapy. In some embodiments, the cancer therapy excludes a systemic cancer therapy. In some embodiments, the cancer therapy excludes a local cancer therapy.

A. Chemotherapies

In some embodiments, the one or more cancer therapies comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane).

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent.

The chemotherapeutics of the disclosure can be tested in vivo for the desired therapeutic activity alone or in combination with another cytotoxic agent, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

1. Anthracene Derivatives

In some embodiments, the chemotherapy comprises an anthracene derivative. In some embodiments, the disclosed methods comprise administration of an anthracene derivative to a subject or patient in need thereof. In some embodiments, the anthracene derivative is bisantrene.

Bisantrene is a tricyclic aromatic compound with the chemical name 9,10-anthracenedicarboxaldehyde bis[(4,5-dihydro-1H-imidazol-2-yl)hydrazine] dihydrochloride. The molecular formula is C₂₂H₂₂N₈·2HCl, and the molecular weight is 471.4. The structure of bisantrene hydrochloride is shown in Formula (I):

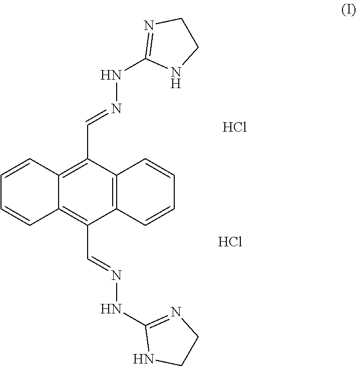 [SMILES: C(=N\NC1=NCCN1)/c1c2ccccc2c(/C=N/NC2=NCCN2)c2ccccc12 ; Cl ; Cl]

The alkylimidazole side chains of bisantrene are basic and, at physiologic pH, are positively charged, which is believed to facilitate electrostatic attractions between bisantrene and the negatively charged ribose phosphate groups in DNA and RNA.

Bisantrene was originally classified as an anthracycline chemotherapeutic agent. Anthracyclines are drugs with planar structures based around a resonant aromatic ring structure that intercalate within the helices of DNA and RNA and disrupt various functions, including DNA and RNA synthesis, presumably due to a strong inhibitory effect on the enzyme topoisomerase II. Anthracyclines, despite their clinical utility, are known to be cardiotoxic. Toxicity studies in dogs and monkeys demonstrated that at high doses, anthracyclines cause leukopenia, anorexia, diarrhea, injection site necrosis, enterocolitis, muscle degeneration, and pulmonary edema. The toxicity of bisantrene, however, has been shown to be than that of the anthracycline doxorubicin. The drug was not cardiotoxic in animals, and use in the clinic has confirmed less cardiotoxicity than other agents in its class. No patients experienced electrocardiographic changes while receiving the drug and radioangiocardiographic monitoring demonstrated no decrease in ejection fraction or any other significant change in cardiac function (J. W. Myers et al., “Radioangiocardiographic Monitoring in Patients Receiving Bisantrene,” Am. J. Clin. Oncol. 7: 129-130 (1984), incorporated herein by this reference). Bisantrene has also been reported to produce very little nausea or vomiting, and alopecia is also less intense with bisantrene compared with doxorubicin (J. D. Cowan et al., “Randomized Trial of Doxorubicin, Bisantrene, and Mitoxantrone in Advanced Breast Cancer: A Southwest Oncology Group Study,” J. Nat'l Cancer Inst. 83: 1077-1084 (1991)).

Among the types of cancer for which a response to bisantrene has been seen are bladder carcinoma, multiple myeloma, lung adenocarcinoma, melanoma, and renal cell carcinoma (Alberts et al. (1982), supra), as well as breast cancer (Bowden et al. (1985), supra) and acute myelogenous leukemia, especially relapsed or refractory acute myeloid leukemia (A. Spadea et al., “Bisantrene in Relapsed and Refractory Myelogenous Leukemia,” Leukemia Lymphoma 9: 217-220 (1993). Bisantrene has also been shown to be effective in cancer models using colon 26, Lewis lung, Ridgway osteosarcoma, B16, Lieberman plasma cell, P388 or L1210 cancer cells. For example, bisantrene has shown antitumor activity in murine tumor models including P-388 leukemia and B-16 melanoma (R. V. Citarella et al., “Anti-Tumor Activity of CL-216942: 9,10-Anthracenedicarboxaldehyde bis [(4,5-dihydro-1H-imidazol-2-yl)hydrazone)]dihydrochloride (Abstract #23) in Abstracts of the 20th Interscience Conference on Antimicrobial Agents and Chemotherapy (Bethesda, Md., American Society for Microbiology 1980)). Human tumor cells that were sensitive to bisantrene as assessed by in vitro colony-forming assays include breast cancer, ovarian cancer, renal cancer, small cell, non-small cell, and squamous cell lung cancer, lymphoma, acute myelogenous leukemia, melanoma, pancreatic cancer, gastric cancer, adrenal cancer, sarcoma, and head and neck cancer (D. D. Von Hoff et al, “Activity of 9,10-Anthracenedicarboxaldehyde bis[(4,5-dihydro-1H-imidazol-2-yl)hydrazine]dihydrochloride (CL216,942) in a human tumor cloning system,” Cancer Chemother. Pharmacol. 6: 141-144 (1981) (“Von Hoff et al. (1981a)”).

In phase I clinical trials, bisantrene showed activity in hepatocellular cancer and hypernephroma (D. D. Von Hoff et al., Phase I Clinical Investigation of 10-Anthracenedicarboxaldehyde bis[(4,5-dihydro-1H-imidazol-2-yl)hydrazine]dihydrochloride (CL216,942),” Cancer Res. 3118-3121 (1981) (“Von Hoff et al. (1981b)”) and in lymphoma, myeloma, melanoma, renal cancer, and tumors of the bladder and lung (D. S. Alberts et al., “Phase I Clinical Investigation of 9,10-Anthracenedicarboxaldehyde bis[(4,5-dihydro-1H-imidazol-2-yl)hydrazone] Dihydrochloride with Correlative in Vitro Human Tumor Clonogenic Assay,” Cancer Res. 42: 1170-1175 (1982)). Phase I activity was also observed in two other hypernephroma patients (R. J. Spiegel et al., “Phase I Clinical Trial of 9,10-Anthracene Dicarboxaldehyde (Bisantrene) Administered in a Five-Day Schedule,” Cancer Res. 42: 354-358 (1982)).

In Phase II clinical trials, bisantrene was active in patients with metastatic breast cancer (H.-Y. Yap et al., “Bisantrene, an Active New Drug in the Treatment of Metastatic Breast Cancer,” Cancer Res. 43: 1402-1404 (1983)). Partial response rates were also observed in heavily pretreated patients with metastatic breast cancer; however, the study was terminated because of significant local toxicity.

Bisantrene doses have been infused via central venous access devices over 1 hour (Van Hoff et al., 1981b). Bisantrene has also been infused through peripheral veins over 2 hours, and has been “piggybacked” into a running dextrose infusion in an attempt to lessen delayed swelling in the arm used for infusion. To reduce venous irritation, hyperpigmentation, drug extravasation, and anaphylactoid reactions, patients have been given hydrocortisone (50 mg IV) and the antihistamine diphenhydramine (50 mg 1M) immediately prior to bisantrene (Alberts et al. (1982)), supra). In one alternative, bisantrene vials have been reconstituted with 2 to 5 mL of Sterile Water for Injection, USP, and then diluted with approximately 0.1 to 0.5 mg/mL in D5W (5% dextrose in water).

Maximally tolerated doses in several bisantrene phase I schedules include: (1) 200 mg/m² weekly×3 (150 mg/mg² for patients with poor bone marrow reserve (e.g., those patients who have received radiotherapy or extensive chemotherapy regimens)) (Alberts et al. (1982), supra); (2) 150 mg/m² weekly×3 (repeat every 4-5 week) (B.-S. Yap et al., “Phase I Clinical Evaluation of 9,10-Anthracenedicarboxaldehyde[bis(4,5-dihydro-1H-imidazol-2-yl)hydrazone]dihydrochloride (Bisantrene),” Cancer Treat. Rep. 66: 1517-1520 (1982)); (3) 260 mg/m² monthly (every 3-4 week) (240 mg/m² for patients with poor bone marrow reserve (e.g., those patients who have received radiotherapy or extensive chemotherapy regimens)) (Von Hoff et al., 1981 b); and (4) 80 mg/m² daily×5 (repeat every 4 weeks) (R. J. Spiegel et al. (1982), supra).

More than 95% of bisantrene is bound to plasma proteins, and the drug has a long terminal plasma half-life. There appeared to be three phases of elimination: an initial distributive phase of 6 minutes, a beta phase of approximately 1.5 hours, and a final gamma elimination phase of 23 to 54 hours (Alberts et al. (1983), supra). Typical areas under the plasma concentration x time curve are 4.4 to 5.7 mg h/mL following intravenous doses of 260 to 340 mg/m², respectively (Alberts et al. 1983, supra). Less than 7% of a bisantrene dose is excreted in the urine; the majority of the drug is eliminated by the hepatobiliary route. The drug may be metabolized to some extent in vivo. In vitro, bisantrene is a substrate for hepatic microsomal enzymes, but specific metabolites have not been identified. Preclinical drug distribution studies have shown that the tissues with the highest concentration (in descending order) are kidney, liver, gallbladder, spleen, lung, and heart. The drug also distributes to lymph nodes and bone marrow (W. H. Wu & G. Nicolau, “Disposition and Metabolic Profile of a New Antitumor Agent, CL 216,942 (Bisantrene) in Laboratory Animals,” Cancer Treat Rep. 66: 1173-1185 (1982)).

Several other clinical trials have also investigated the pharmacokinetics of bisantrene in humans. In one trial, patients were given a 90 min infusion of bisantrene at 260 mg/m², a biphasic elimination with an initial half-life of 65±15 min, a terminal half-life of 1142±226 min, and a steady state volume of distribution (Vdss) of 1845 L/m² was observed. Plasma clearance in this trial was 735 ml/min/m², with 11.3% of the administered dose excreted unchanged in the urine after 24 hr. In another trial, doses of 80-250 mg/m² were assessed, and the initial and terminal half-lives were 0.6 hr and 24.7 hr, respectively, with a clearance of 1045.5±51.0 ml/kg/hr and a calculated volume of distribution of 42.1±5.9 L/kg. In this study, only 3.4±1.1% of the administered dose was found in the urine after 96 hr. Triphasic elimination has also been reported for three other single dose studies. The first of the three studies reported a t½ α, β, and γ of 3.44 min, 1.33 hr, and 26.13 hr, respectively; the second of the three studies reported a t½ α, β, and γ of 3 min, 1 hr, and 8 hr respectively; and the last of the three studies reported a t½ α, β, and γ of 0.1 hr, 1.9 hr and 43.9 hr, respectively. In one report, a large volume of distribution (687 L/m²) was interpreted as tissue sequestration of the drug with a subsequent depot effect. In a 72-hr infusion study, a plasma concentration of 12±6 ng/ml was observed at a dose of 56 mg/m², while a dose of 260 mg/m² resulted in a plasma concentration of 55±8 ng/ml. In this trial, plasma clearance was 1306±179 ml/min/m², with urinary excretion of 4.6% of the dose in 24 hr. Finally, in another study, a 5 day schedule of 60 min infusions revealed a t½ α and β of 0.9 and 9.7 hr, respectively, with 7.1% of the dose excreted in the urine.

Various formulations suitable for use in the administration of bisantrene or derivatives or analogs thereof are known in the art. U.S. Pat. No. 4,784,845 to Desai et al., incorporated by reference herein in its entirety, discloses a composition of matter for delivery of a hydrophobic drug (i.e., bisantrene or a derivative or analog thereof) comprising: (i) the hydrophobic drug; (ii) an oleaginous vehicle or oil phase that is substantially free of butylated hydroxyanisole (BHA) or butylated hydroxytoluene (BHT); (iii) a co-surfactant or emulsifier; (iv) a co-surfactant or auxiliary emulsifier; and (v) benzyl alcohol as a co-solvent. U.S. Pat. No. 4,816,247 by Desai et al., incorporated by reference herein in its entirety, discloses a composition of matter for delivery by intravenous, intramuscular, or intraarticular routes of hydrophobic drugs (such as bisantrene or a derivative or analog thereof) comprising: (i) the hydrophobic drug; (ii) a pharmaceutically acceptable oleaginous vehicle or oil selected from the group consisting of: (a) naturally occurring vegetable oils and (b) semisynthetic mono-, di-, and triglycerides, wherein the oleaginous vehicle or oil is free of BHT or BHA; (iii) a surfactant or emulsifier; (iv) a co-surfactant or emulsifier; (v) an ion-pair former selected from C₆-C₂₀ saturated or unsaturated aliphatic acids when the hydrophobic drug is basic and a pharmaceutically acceptable aromatic amine when the hydrophobic drug is acidic; and (vi) water. U.S. Pat. No. 5,000,886 to Lawter et al. and U.S. Pat. No. 5,143,661 to Lawter et al., each incorporated by reference herein in its entirety, disclose compositions for delivery of pharmaceutical agents such as bisantrene or a derivative or analog thereof comprising a microcapsule, wherein the microcapsule includes a hardening agent that is a volatile silicone fluid. U.S. Pat. No. 5,070,082 to Murdock et al., U.S. Pat. No. 5,077,282 to Murdock et al., and U.S. Pat. No. 5,077,283 to Murdock et al., all incorporated by reference herein in their entirety, disclose prodrug forms of poorly soluble hydrophobic drugs, including bisantrene and derivatives and analogs, that are salts of a phosphoramidic acid. U.S. Pat. No. 5,116,827 to Murdock et al. and U.S. Pat. No. 5,212,291 to Murdock et al., each incorporated by reference herein in its entirety, disclose prodrug forms of poorly soluble hydrophobic drugs, including bisantrene and derivatives and analogs, that are quinolinecarboxylic acid derivatives. U.S. Pat. No. 5,378,456 to Tsou, incorporated by reference herein in its entirety, includes compositions containing an anthracene antitumor agent, such as bisantrene or a derivative or analog thereof, in which the bisantrene or derivative or analog thereof is conjugated to or admixed with a divinyl ether-maleic acid (MVE) copolymer. U.S. Pat. No. 5,609,867 to Tsou, incorporated by reference herein in its entirety, discloses polymeric 1,4-bis derivatives of bisantrene and copolymers of bisantrene and another monomer, such as a dianhydride.

Improved methods and compositions for the use of bisantrene and analogs or derivatives thereof for the treatment of cancer while improving the therapeutic efficacy, stability, and/or bioavailability of the drug and avoiding side effects of bisantrene treatment, including myelosuppression, leukopenia, anaphylactoid reactions, phlebitis, erythema, and edema, are described in U.S. Pat. No. 9,974,774, PCT App. No. WO 2019/073296, and PCT App. No. 2020/072948, each incorporated by reference herein in its entirety. These patents and applications describe novel compositions and methods to improve the utility of chemical agents including bisantrene and derivatives and analogs thereof, as described above, with suboptimal performance for patients with cancer and the novel development of improved pharmaceutical ingredients, dosage forms, excipients, solvents, diluents, drug delivery systems, preservatives, more accurate drug administrations, improved dose determination and schedules, toxicity monitoring and amelioration, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches, the use of drug delivery systems, novel prodrugs, polymer conjugates, novel routes of administration, other agents to potentiate the activity of the compounds or inhibit the repair of suboptimal cellular effects or sub-lethal damage or to “push” the cell into more destructive cellular phases such as apoptosis. In some cases, the inventive examples include the use of these sub-optimal therapeutics in conjunction with radiation or other conventional chemotherapeutic agents or biotherapeutic agents such as antibodies, vaccines, cytokines, lymphokines, gene and antisense therapies, or other biotherapeutic agents.

Bisantrene has been found to have direct cytotoxic action and non-immunologic effects. The noncovalent binding of bisantrene to DNA comprises two types of interactions: (1) intercalation of the planar anthracene moiety between DNA base pairs; and (2) electrostatic binding between negatively charged ribose phosphates of DNA and positively charged basic nitrogens on the alkyl side chains of the drug. This is reflected in the biphasic DNA dissociation curves for bisantrene in calf thymus DNA in vitro (W. O. Foye et al., “DNA-Binding Abilities of Bisguanylhydrazones of anthracene-9,10-dicarboxaldehyde,” Anti-Cancer Drug Design 1:65-71 (1986)). Like other anthracyclines, bisantrene intercalates with DNA, preferentially binding to A-T rich regions where it effects changes to supercoiling and initiates strand breaks in association with DNA associated proteins. This results from the inhibition of topoisomerase II, which relaxes DNA coiling during replication. Specifically, bisantrene has been shown to induce altered DNA supercoiling indicative of DNA intercalation (G. T. Bowden et al., “Comparative Molecular Pharmacology in Leukemic L1210 cells of the Anthracene Anticancer Drugs Mitoxantrone and Bisantrene, Cancer Res. 45: 4915-4920 (1985)). In L-1210 leukemia cells, bisantrene was also shown to induce protein-associated DNA strand breaks typical of drug-induced inhibition of DNA topoisomerase II enzymes (Bowden et al., 1985). Both cytotoxicity and DNA strand breaks appear to be reduced in hypoxic conditions (C. U. Ludwig et al., “Reduced Bisantrene-Induced Cytotoxicity and Protein-Associated DNA Strand Breaks Under Hypoxic Condition,” Cancer Treat. Rep. 68: 367-372 (1984)).

In addition to direct antineoplastic effects related to the activity of bisantrene as a DNA intercalator, bisantrene also possesses other mechanisms of action, including genomic and immunologic modes of action, such as immunopotentiation. For example, bisantrene has been reported as activating tumor-cytostatic macrophages (B. S. Wang et al., “Activation of Tumor-Cytostatic Macrophages with the Antitumor Agent 9,10-Anthracenedicarboxaldehyde Bis[(4,5-dihydro-1H-imidazole-2-yl)hydrazine Dihydrochloride (Bisantrene),” Cancer Res. 44: 2363-2367 (1984)), incorporated herein by this reference. The minimal effective in vivo dose of bisantrene appeared to be 25 mg/kg, with peak activation being achieved at doses of 50 to 100 mg/kg. The efficacy of bisantrene in allogeneic macrophage transplants and with supernatants of macrophages activated by bisantrene has been shown in B. S. Wang et al., “Immunotherapy of a Murine Lymphoma by Adoptive Transfer of Syngeneic Macrophages Activated by Bisantrene,” Cancer Res. 46: 503-506 (1986), incorporated herein by reference. Specifically, the active cells were obtained from peritoneal exudate. Bisantrene-activated macrophages were shown to be highly cytostatic to tumor cells. Repeated treatments with activated macrophages were shown to be more effective in protecting animals inoculated with tumors. This represents immunotherapy by adoptive transfer of immunocompetent cells. Culture supernatants of activated macrophages were also found to have antiproliferative effects on tumor cells, indicating that a cytostatic factor or factors were produced by these macrophages. (B. S. Wang et al., “Activation of Tumor-Cytostatic Macrophages with the Antitumor Agent 9,10-Anthracenedicarboxaldehyde Bis[(4,5-dihydro-1H-imidazole-2-yl)hydrazine] Dihydrochloride (Bisantrene),” Cancer Res. 44: 2363-2367 (1984)). Thus, the supernatants from bisantrene-activated macrophages have a protective cytostatic effect in tumor cell cultures, and bisantrene-activated macrophages adoptively transferred to mice with EL-4 lymphomas more than doubled the median survival time of the mice, with 7 of 10 mice in the group being cured. There is also evidence that inhibition of DNA expression by bisantrene mimics the effects of survivin inhibitors including NSC80467 and YM155.

Other immunological mechanisms are described in: (i) N. R. West et al., “Tumor-Infiltrating Lymphocytes Predict Response to Anthracycline-Based Chemotherapy in Estrogen-Resistant Breast Cancer,” Breast Canc. Res. 13: R126 (2011), which concludes that the level of tumor-infiltrating lymphocytes is correlated with a response to the administration of anthracycline-based agents; the markers associated with tumor-infiltrating lymphocytes (TIL) include CD19, CD3D, CD48, GZMB, LCK, MS4A1, PRF1, and SELL; (ii) L. Zitvogel et al., “Immunological Aspects of Cancer Chemotherapy,” Nature Rev. Immunol. 8: 59-73 (2008), which states that DNA damage, such as that produced by intercalating agents such as bisantrene, induces the expression of NKG2D ligands on tumor cells in an ATM-dependent and CHK1-dependent (but p53-independent) manner; NKG2D is an activating receptor that is involved in tumor immunosurveillance by NK cells, NKT cells, 76 T cells and resting (in mice) and/or activated (in humans) CD8+ T cells, and also states that anthracycline-based agents may act as immunostimulators, particularly in combination with IL-12; such agents also promote HMGB1 release and activate T cells; (iii) D. V. Krysko et al., “TLR2 and TLR9 Are Sensors of Apoptosis in a Mouse Model of Doxorubicin-Induced Acute Inflammation,” Cell Death Different. 18: 1316-1325 (2011), which states that anthracycline-based antibiotics induce an immunogenic form of apoptosis that has immunostimulatory properties mediated by MyD88, TLR2, and TLR9; (iv) C. Ferraro et al., “Anthracyclines Trigger Apoptosis of Both G0-G1 and Cycling Peripheral Blood Lymphocytes and Induce Massive Deletion of Mature T and B Cells,” Cancer Res. 60: 1901-1907 (2000), which stated that anthracyclines induce apoptosis and ceramide production, as well as activate caspase-3 in resting and cycling cells; the apoptosis induced is independent from CD95-L/CD95 and TNF/TNF-R; and (v) K. Lee et al., “Anthracycline Chemotherapy Inhibits HIF-1 Transcriptional Activity and Tumor-Induced Mobilization of Circulating Angiogenic Cells,” Proc. Natl. Acad. Sci USA 106: 2353-2358 (2009), which provides another antineoplastic mechanism for anthracycline-based antibiotics, namely inhibition of HIF-1 mediated gene transcription, which, in turn, inhibits transcription of VEGF required for angiogenesis; HIF-1 also activates transcription of genes encoding glucose transporter GLUT1 and hexokinases HK1 and HK2, which are required for the high level of glucose uptake and phosphorylation that is observed in metastatic cancer cells, and pyruvate dehydrogenase kinase 1 (PDK1), which shunts pyruvate away from the mitochondria, thereby increasing lactate production; patients with HIF-1α overexpression based on immunohistochemical results were suggested to be good candidates for treatment with anthracycline-based antibiotics.

Analogs of bisantrene have also been made in an attempt to improve its anti-telomerase activity. Human melanoma (SK-Mel5) and colon cancer (LoVo) tumor cells were observed to lose their proliferative ability in the presence of these analogs. Though apoptosis was not observed, a loss of immortality was demonstrated, with treated cells reacquiring the ability to become senescent, age, and die.

Bisantrene and analogs thereof have been reported as inhibiting telomerase activity, especially by stabilizing G-quadruplex DNA structures formed at sites where four guanines associate by folding, as disclosed in M. Folini et al., “Remarkable Interference with Telomeric Function by a G-Quadruplex Selective Bisantrene Regioisomer,” Biochem. Pharmacol. 79: 1781-1790 (2010), incorporated by reference herein in its entirety. Telomerase is a ribonucleoprotein reverse transcriptase responsible for maintenance of telomere length. Its expression is associated with cell immortalization and tumorigenesis since it is expressed in most human tumor cells but is not active in most normal somatic cells. Generally, inhibition of telomerase activity results in cellular senescence or apoptosis in a time-dependent manner that correlates with the initial telomere length in the cells in which telomerase is inhibited. When telomere architecture collapses or is disrupted, a signaling cascade comparable to that produced by DNA damage is activated, and cell cycle arrest (accelerated senescence) or cell death through apoptosis is induced.

Telomerase substrates are the telomeres, double-stranded DNA portions with a 3′ protruding overhang (100-200 bases long), formed by a repeating noncoding sequence (TTAGGG in humans). The single-stranded portion can fold into a structure called G-quadruplex, overlapping planar regions formed from four Hoogsteen-paired guanines. Hoogsteen base-pairing is between the N7 position of the purine base as a hydrogen-bond acceptor and the C6 amino group of the pyrimidine base as a donor. By recognizing and stabilizing this abnormal DNA base-pairing arrangement, selected ligands like bisantrene impair telomere-telomerase interactions, thus interfering with the telomere elongation step catalyzed by the enzyme. Additionally, bisantrene can displace the telomere binding proteins (i.e., TRF2 and hPOT1) involved in telomere capping, thereby allowing recognition of the free terminal sequence as a DNA damage region. Compounds like bisantrene able to interact with and stabilize G-quadruplex structures formed by G-rich single-stranded overhangs of telomeres share a general consensus structural motif based on a large flat aromatic surface linked to protonatable side chains. DNA binding occurs mainly through stacking on a terminal G-tetrad, whereas side chains contribute to the stability of the complex by hydrophobic/ionic interactions with the DNA grooves.

Bisantrene analogs include but are not limited to those of Formulas (II), (III), (IV), (V), (VI), (VII), and (VIII):

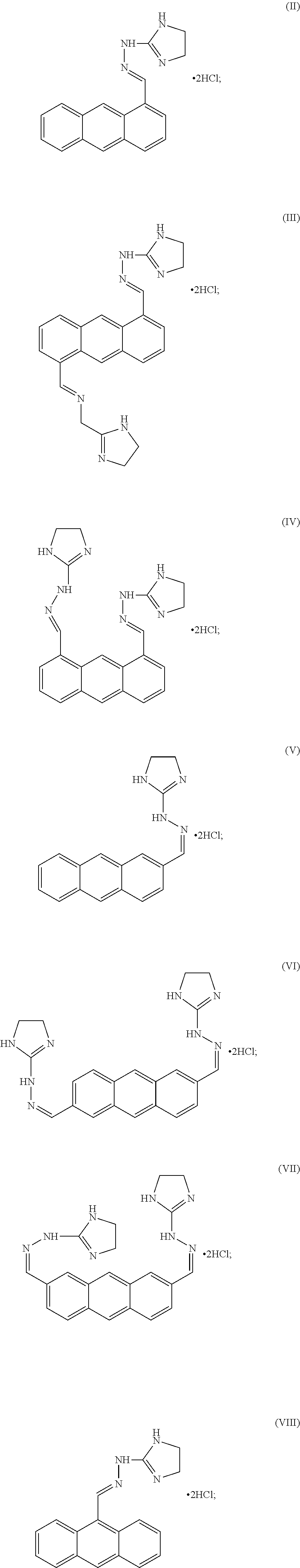

Since similar basic features characterize intercalation and base stacking, the scaffolds of classical intercalating agents are commonly used as starting structures to produce compounds that recognize and bind to G-quadruplexes. At least two side chains with amine groups protonatable at physiological pH are required for G-quadruplex binding. The most efficient G-quadruplex binders are substituted on two distinct aromatic rings with side chains pointing in opposite directions with reference to the long axis of the aromatic system likely suggests formation of additional specific interactions between the 4,5-dihydro-1H-imidazol-2-yl hydrazone groups and the G-quadruplex structure.

Bisantrene shares the structural “consensus motif” characteristic of effective G-quadruplex binders. Bisantrene is believed to intercalate between adjacent base pairs of double-stranded DNA through π-π stacking, with side chains located in either groove (threading mode), which grants affinity constants well above 10⁶ M⁻¹ under physiological conditions. At least one of the bisantrene analogs, Formula (III), has the ability to act both at the telomerase level by interfering with substrate recognition and suppressing catalytic activity and at the telomere level by modifying structural organization of telomeres. This compound affects telomere function not only in telomerase-expressing cells but also in ALT-positive cell lines, since it consistently provokes a DNA damage response, as evidenced by the formation of γH2AX foci that partially co-localize at the telomere, in agreement with results reported for telomestatin. For this compound, such a DNA damage response, together with the absence of apoptosis and the induction of cell cycle impairment (mainly G2M phase arrest), suggest a drug-mediated activation of a senescence pathway.

Another bisantrene analog is the compound known as HL-37 and described in S. Q. Xie et al., “Anti-Tumour Effects of HL-37, a Novel Anthracene Derivative, In-Vivo and In-Vitro,” J. Pharm. Pharmacol. 60:213-219 (2008), incorporated by reference herein in its entirety. HL-37 is anthracen-9-ylmethylene-[2-methoxyethoxymethylsulfanyl]-5-pyridin-3-yl-[1,2,4]triazol-4-amine and has the structure shown below as Formula (IX):

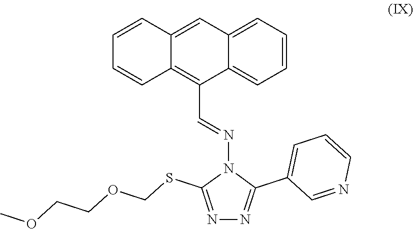 [SMILES: COCCOCSc1nnc(-c2cccnc2)n1/N=C/c1c2ccccc2cc2ccccc12]

Other bisantrene analogs and derivatives are known in the art, including the bisantrene analogs disclosed in T. P. Wunz et al., “New Antitumor Agents Containing the Anthracene Nucleus,” J. Med. Chem. 30: 1313-1321 (1987), including N,N′-bis[2-(dimethylamino)ethyl]-9,10-anthracene-bis(methylamine) and N,N′-bis(1-ethyl-3-piperidinyl)-9,10-anthracene-bis(methylamine), and in J. A. Elliott et al., “Interaction of Bisantrene Anti-Cancer Agents with DNA: Footprinting, Structural Requirements for DNA Unwinding, Kinetics and Mechanism of Binding and Correlation of Structural and Kinetic Parameters with Anti-Cancer Activity,” Anticancer Drug Dis. 3: 271-282 (1989). In C. Sissi et al., “DNA-Binding Preferences of Bisantrene Analogs: Relevance to the Sequence Specificity of Drug-Mediated Topoisomerase II Poisoning,” Mol. Pharmacol. 54: 1036-1045 (1998) discloses additional analogs, including an aza-bioisostere that can be considered a bisantrene-amsacrine hybrid. Still other bisantrene analogs and derivatives are disclosed in G. Zagotto et al., “Synthesis, DNA-Damaging and Cytotoxic Properties of Novel Topoisomerase II-Directed Bisantrene Analogues,” Bioorg. Med. Chem. Lett. 20: 121-126 (1998). T. L. Fields et al., “The Synthesis of Heterocyclic Analogs of Bisantrene,” J. Heterocyclic Chem. 25: 1917-1918 (1988) discloses bisguanylhydrazones of anthracene-9,10-dicarboxaldehyde as bisantrene analogs. Bisantrene-amsacrine hybrids are also disclosed in G. Capranico et al., “Mapping Drug Interactions at the Covalent Topoisomerase II-DNA Complex by Bisantrene/Amsacrine Congeners,” J. Biol. Chem. 273: 12732-12739 (1998). These compounds are depicted below as Formulas (X), (XI), (XII), and (XIII):

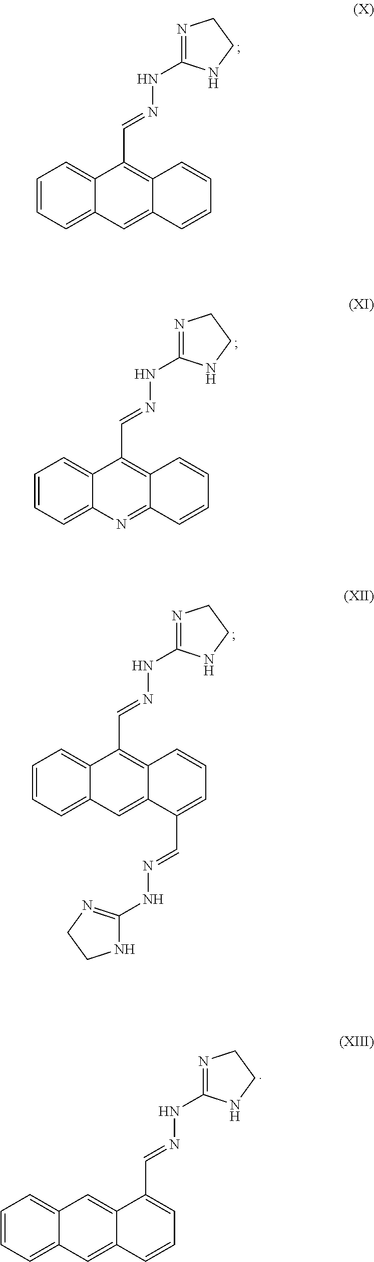 [SMILES: C(=N\NC1=NCCN1)/c1c2ccccc2cc2ccccc12 ; C(=N\NC1=NCCN1)/c1c2ccccc2nc2ccccc12 ; C(=N\NC1=NCCN1)/c1cccc2c(/C=N/NC3=NCCN3)c3ccccc3cc12 ; C(=N\NC1=NCCN1)/c1cccc2cc3ccccc3cc12]

Additional derivatives and analogs of bisantrene include the diphosphoramidic and monophosphoramidic derivatives of bisantrene, disclosed in U.S. Pat. No. 4,900,838 to Murdock and U.S. Pat. No. 5,212,191 to Murdock et al., both of which are incorporated herein by this reference. These compounds are compounds of Formula (XIV):

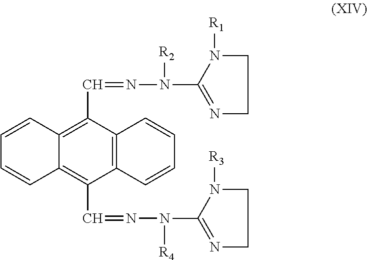

wherein R₁ an 3 are the same or different and are hydrogen, C₁-C₆ alkyl, C(O)—R₅, wherein R₅ is hydrogen, C₁-C₆ alkyl, phenyl, mono-substituted phenyl (wherein the substituent can be ortho, meta, or para and is fluoro, nitro, C₁-C₆ alkyl, C₁-C₃ alkoxy, or cyano), entafluorophenyl, naphthyl, furanyl,

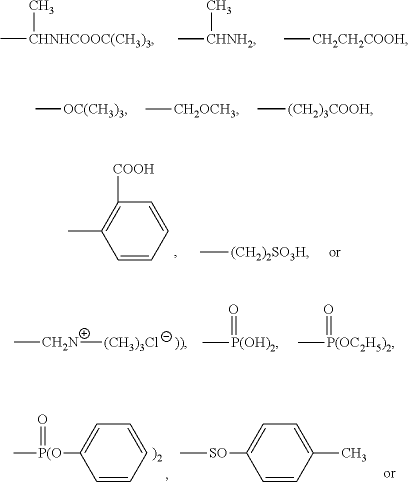

—SO₃H; wherein only one of R₁ and R₃ may be hydrogen or C₁-C₆ alkyl; R₂ and R₄ are the same or different and are: hydrogen, C₁-C₄ alkyl or —C(O)—R₆, where R₆ is hydrogen, C₁-C₆ alkyl, phenyl, mono-substituted phenyl (wherein the substituent may be in the ortho, meta, or para position and is fluoro, nitro, C₁-C₆ alkyl, C₁-C₃ alkoxy, or cyano), pentafluorophenyl, naphthyl, furanyl, or —CH₂OCH₃. The compounds can have the schematic structure B(Q)ₙ, wherein B is the residue formed by removal of a hydrogen atom from one or more basic nitrogen atoms of an amine, amidine, guanidine, isourea, isothiourea, or biguanide-containing pharmaceutically active compound, and Q is hydrogen or A, wherein A is

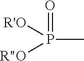

such that R′ and R″ are the same or different and are R (where R is C₁-C₆ alkyl, aryl, aralkyl, heteroalkyl, NC—CH₂CH₂—,

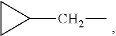

Cl₃C—C₂—, or R₇OCH₂CH₂—, where R₇ is hydrogen or C₁-C₆ alkyl, hydrogen, or a pharmaceutically acceptable cation or R′ and R″ are linked to form a CH₂CH₂ group or a

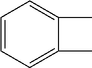

group, and n is an integer representing the number of primary or secondary basic nitrogen atoms in the compound such that at least one Q is A.

Additional bisantrene analogs are disclosed in M. Kozurková et al., “DNA Binding Properties and Evaluation of Cytotoxic Activity of 9,10-Bis-N-Substituted (Aminomethyl)anthracenes,” Int. J. Biol. Macromol. 41: 415-422 (2007). These compounds include 9,10-bis[(2-hydroxyethyl)iminomethyl]anthracene; 9,10-bis{[2-(-2-hydroxyethylamino)ethyl]iminomethyl}anthracene; 9,10-bis{[2-(morpholin-4-yl)ethyl]iminomethyl}anthracene; 9,10-bis[(2-hydroxyethyl)aminomethyl]anthracene; 9,10-bis{[2-(2-hydroxyethylamino)ethyl]aminomethyl}anthracene tetrahydrochloride; 9,10-bis{[2-(piperazin-1-yl)ethyl]aminomethyl}anthracene hexahydrochloride; and 9,10-bis{[2-(morpholin-4-yl)ethyl]aminomethyl}anthracene tetrahydrochloride.

Other analogs and derivatives are known in the art, including derivatives and salt forms of the compounds described above.

Bisantrene and its analogs and derivatives possess antineoplastic activity through several mechanisms, including, but not necessarily limited to, intercalation in DNA, inhibition of the enzyme topoisomerase II, immune stimulation, and inhibition of telomerase. These activities are described above. Also, as described above, bisantrene and its analogs and derivatives also can activate macrophages.

As used herein, the term “derivative” as applied to bisantrene refers to a compound that has the same carbon skeleton as bisantrene, including the tricyclic aromatic nucleus and the two side chains attached to the tricyclic aromatic nucleus but has one or more substituents as described below that replace at least one hydrogen present in bisantrene with another moiety. As used herein, the term “analog” as applied to bisantrene applies to a compound related structurally to bisantrene but having one or more of the tricyclic aromatic nucleus or one or more of the side chains altered, for example, by replacing one or more carbons in the tricyclic aromatic nucleus with nitrogens or by removing or moving one or both of the side chains. Some analogs are described above; others are known to one of skill in the art.

In summary, bisantrene and its derivatives or analogs can be expected to have antineoplastic activity against the following cancers: breast cancer, ovarian cancer, renal cancer, small-cell lung cancer, non-small cell lung cancer, Hodgkin's lymphoma, non-Hodgkin's lymphoma, acute myelocytic leukemia, acute lymphocytic leukemia, melanoma, gastric cancer, adrenal cancer, head and neck cancer, hepatocellular cancer, hypernephroma, bladder cancer, acute leukemias of childhood, chronic lymphocytic leukemia, prostate cancer, glioblastoma, and myeloma. Thus, the cancer treated by the methods of some embodiments is breast cancer, ovarian cancer, renal cancer, small-cell lung cancer, non-small cell lung cancer, Hodgkin's lymphoma, non-Hodgkin's lymphoma, acute myelocytic leukemia, acute lymphocytic leukemia, melanoma, gastric cancer, adrenal cancer, head and neck cancer, hepatocellular cancer, hypernephroma, bladder cancer, acute leukemias of childhood, chronic lymphocytic leukemia, prostate cancer, glioblastoma, and myeloma.

Derivatives of bisantrene include, but are not limited to: (1) derivatives of bisantrene in which at least one of the hydrogen atoms bound to the carbon atoms that are directly bound to the tricyclic aromatic nucleus is replaced with lower alkyl; (2) derivatives of bisantrene in which at least one of the hydrogen atoms in the N═NH moiety is replaced with lower alkyl; or (3) derivatives of bisantrene in which at least one of the hydrogen atoms bound to the nitrogens of the five-membered rings are replaced with lower alkyl. Other derivatives of bisantrene are described below.

Analogs of bisantrene include, but are not limited to compounds described above as Formulas (II)-(XIV), as well as additional compounds described above and their derivatives.

As described above, and as detailed more generally below, derivatives and analogs of bisantrene can be optionally substituted with one or more groups that do not substantially affect the pharmacological activity of the derivative or analog. These groups are generally known in the art. Definitions for a number of common groups that can be used as optional substituents are provided below; however, the omission of any group from these definitions should not be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds.

As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included.

As used herein, the term “aryl” refers to a monocyclic or fused bicyclic moiety having the well-known characteristics of aromaticity; examples include phenyl and naphthyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicyclic ring systems that have the characteristics of aromaticity and include one or more heteroatoms selected from O, S, and N.

The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition.

This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation.

As used herein, the term “hydroxheteroaryl” refers to a heteroaryl group including one or more hydroxyl groups as substituents; as further detailed below, further substituents can be optionally included.

As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S—, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O—, —S(O₂)OZ^{b}, —OS(O₂)OZ^{b}, —OS(O₂)O—, —OS(O₂)OZ^{b}, —P(O)(O—)₂, —P(O)(OZ^{b})(O—), —P(O)(OZ^{b})(OZ^{b}), —C(O)Z^{b}, —C(S)Z^{b}, —C(NZ^{b})Z^{b}, —C(O)O—, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O—, —OC(O)OZ^{b}, —OC(S)OZ^{b}, —NZ^{b}C(O)Z^{b}, —NZ^{b}C(S)Z^{b}, —NZ^{b}C(O)O—, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c}s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, NZ^{c}Z^{c} is meant to include NH₂, —NH-alkyl, N-pyrrolidinyl, and N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and CH₂—CH₂—C(O)—CH₃₃ but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, -halo, —O—, —OZ^{b}, —SZ^{b}, —S—, —NZ^{c}Z^{c}, -trihalomethyl, —CF₃, —CN, —OCN, —SCN, —NO, —NO₂, —N₃, —S(O)₂Z^{b}, —S(O₂)—, —S(O₂)OZ^{b}, —OS(O₂)OZ^{b}, —OS(O₂)O—, —P(O)(O—)₂, —P(O)(OZ^{b})(O—), —P(O)(OZ^{b})(OZ^{b}), —C(O)Z^{b}, —C(S)Z^{b}, —C(NZ^{b})Z^{b}, —C(O)O—, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O—, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and NZ^{b}C(NZ^{b})NZ^{c}Z^{c}, wherein Z^{a}, —Z^{b}, and Z^{c} are as defined above.

Similarly, substituent groups useful for substituting nitrogen atoms in heteroalkyl and cycloheteroalkyl groups include, but are not limited to, —Z^{a}, -halo, —O—, —OZ^{b}, —SZ^{b}, —S—, —NZ^{c}Z^{c}, -trihalomethyl, —CF₃, —CN, —OCN, —SCN, —NO, —NO₂, —S(O)₂Z^{b}, —S(O₂)O—, —S(O₂)OZ^{b}, —OS(O₂)OZ^{b}, —OS(O₂)O—, —P(O)(O)₂, —P(O)(OZ^{b})(O), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The disclosure includes each of the isolated stereoisomeric forms (such as the enantiomerically pure isomers, the E and Z isomers, and other stereoisomeric forms) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The disclosure includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the disclosure to the depicted isomer. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the disclosure, with one or more solvent molecules. When water is the solvent, the corresponding solvate is a “hydrate.” Examples of hydrates include, but are not limited to, hemihydrate, monohydrate, dihydrate, trihydrate, hexahydrate, and other hydrated forms. It should be understood by one of ordinary skill in the art that the pharmaceutically acceptable salt and/or prodrug of the present compound may also exist in a solvate form. The solvate is typically formed via hydration which is either part of the preparation of the present compound or through natural absorption of moisture by the anhydrous compound of the present disclosure.

As used herein, the term “ester” means any ester of a present compound in which any of the —COON functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two double bonds, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C₇-arylalkyl group, and phenylethyl is a C₈-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)n- where n is 1-8 and preferably n is 1-4, though where specified, an alkylene can also be substituted by other groups, and can be of other lengths, and the open valences need not be at opposite ends of a chain. The general term “alkylene” encompasses more specific examples such as “ethylene,” wherein n is 2, “propylene,” wherein n is 3, and “butylene,” wherein n is 4. The hydrocarbyl groups of the alkylene can be optionally substituted as described above.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the general term “carbocyclyl” encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur, although, in some contexts, “heteroatom” can refer to phosphorus, selenium, or other atoms other than carbon or hydrogen. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent. As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H. As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al., incorporated herein by this reference. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

Accordingly, methods and compositions according to the present disclosure encompass bisantrene derivatives and analogs including one or more optional substituents as defined above, provided that the optionally substituted bisantrene derivative or analog possesses substantially equivalent pharmacological activity to amonafide as defined in terms of either or both topoisomerase II inhibition and DNA intercalation. Methods for determination of topoisomerase II inhibition are known in the art and are described, for example, in A. Constantinou et al., “Novobiocin- and Phorbol-12-Myristate-13-Acetate-Induced Differentiation of Human Leukemia Cells Associates with a Reduction in Topoisomerase II Activity,” Cancer Res. 49: 1110-1117 (1989), incorporated herein by this reference. Methods for determination of DNA intercalation are known in the art and are described, for example, in H. Zipper et al., “Investigations on DNA Intercalation and Surface Binding by SYBR Green I, Its Structure Determination and Methodological Implications,” Nucl. Acids. Res. 32(12): e103 (2004), incorporated herein by this reference.

2. Pyrimidine Analog Antimetabolites

In some embodiments, the chemotherapy comprises one or more pyrimidine analog antimetabolites. In some embodiments, the disclosed methods comprise administration of one or more pyrimidine analog antimetabolites to a subject or patient in need thereof. As used herein, a “pyrimidine analog antimetabolite” (also “pyrimidine analog” or “antimetabolite”) describes an antimetabolite compound that affects the metabolism and utilization of pyrimidines. In some embodiments, a pyrimidine analog antimetabolite of the present disclosure is cytarabine, fludarabine, cladribine, clofarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, or 2′-C-cyano-2′-deoxy-arabinofuranosylcytosine. In some embodiments, a pyrimidine analog antimetabolite is cytarabine, fludarabine, cladribine, or clofarabine. Embodiments of the disclosure comprise administration of at least 1, 2, 3, 4, 5, or more pyrimidine analog antimetabolites to a subject having cancer. In some embodiments, the one or more pyrimidine analog antimetabolites comprise two or more of cytarabine, fludarabine, cladribine, and clofarabine. In some embodiments, the one or more pyrimidine analog antimetabolites comprise fludarabine and clofarabine.

a. Cytarabine

In some embodiments, the pyrimidine analog antimetabolite is cytarabine. Cytarabine, also known as cytosine arabinoside (ara-C), has the chemical name of 1β-arabinofuranosylcytosine. The structural formula is C₉H₁₃N₃O₅ (M.W. 243.22 g/mol). It is a chemotherapy medication used to treat acute myeloid leukemia (AML), acute lymphocytic leukemia (ALL), chronic myelogenous leukemia (CMIL), and non-Hodgkin's lymphoma, for example. Cytosine arabinoside combines a cytosine base with an arabinose sugar. It is similar enough to human deoxycytosine to be incorporated into human DNA, but dissimilar enough to interfere with the synthesis of DNA. Cytarabine is transported into the cell primarily by hENT-1. It is then monophosphorylated by deoxycytidine kinase and eventually cytarabine-5′-triphosphate, which is the active metabolite incorporated into DNA during DNA synthesis. Its rapid conversion into cytosine arabinoside triphosphate damages DNA when the cell cycle holds in the S phase of DNA synthesis of DNA. Cytosine arabinoside also inhibits both DNA and RNA polymerases and nucleotide reductase enzymes needed for DNA synthesis.

Several mechanisms of resistance to cytarabine have been reported. For example, cytarabine is rapidly deaminated by cytidine deaminase in the serum into the inactive uracil derivative. Cytarabine-5′-monophosphate is deaminated by deoxycytidylate deaminase, leading to the inactive uridine-5′-monophosphate analog. Cytarabine-5′-triphosphate is also a substrate for SAMDH1, and SAMHD1 has been shown to limit the efficacy of cytarabine efficacy in patients.

Cytarabine is often given by continuous intravenous infusion, which follows a biphasic elimination: initial fast clearance rate followed by a slower rate of the analog. There is an initial distributive phase with a half-life of about 10 minutes, followed by a second elimination phase with a half-life of about 1 to 3 hours. After the distributive phase, more than 80% of plasma radioactivity can be accounted for by the inactive metabolite 1-β-D-Arabinofuranosyluracil (ara-U). Within 24 hours, about 80% of the administered radioactivity can be recovered in the urine, approximately 90% of which is excreted as ara-U. Relatively constant plasma levels can be achieved by continuous intravenous infusion. After subcutaneous or intramuscular administration of cytarabine labeled with tritium, peak plasma levels of radioactivity are achieved about 20 to 60 minutes after injection and are considerably lower than those after intravenous administration.

The schedule and method of cytarabine administration varies with the program of therapy to be used. Cytarabine may be given by intravenous infusion or injection, subcutaneously, or intrathecally, for example. Patients can tolerate higher total doses when they receive the drug by rapid intravenous injection as compared with slow infusion. This phenomenon is related to the drug's rapid inactivation and brief exposure of susceptible normal and neoplastic cells to significant levels after rapid injection. Normal and neoplastic cells seem to respond in somewhat parallel fashion to these different modes of administration and no clear-cut clinical advantage has been demonstrated for either.

b. Fludarabine

In some embodiments, the pyrimidine analog antimetabolite is fludarabine. Fludarabine is a fluorinated nucleotide analog of the antiviral agent vidarabine, 9-p-D-arabinofuranosyladenine (ara-A) that is relatively resistant to deamination by adenosine deaminase. The chemical name for fludarabine is 9H-Purin-6-amine, 2-fluoro-9-(5-0-phosphono-0-D-arabino-furanosyl) (2-fluoro-ara-AMP). The molecular formula of fludarabine is C₁₀H₁₃FN₅O₇P (MW 365.2 g/mol). also known as cytosine arabinoside (ara-C), has the chemical name of 1β-arabinofuranosylcytosine. It is a chemotherapy medication used to treat chronic lymphocytic leukemia, non-Hodgkin's lymphoma, acute myeloid leukemia, and acute lymphocytic leukemia, for example.

Fludarabine is rapidly dephosphorylated to 2-fluoro-ara-A and then phosphorylated intracellularly by deoxycytidine kinase to the active triphosphate, 2-fluoro-ara-ATP. This metabolite appears to act by inhibiting DNA polymerase alpha, ribonucleotide reductase and DNA primase, thus inhibiting DNA synthesis primarily in the S-phase of cell division. It is also postulated that fludarabine interferes with RNA by decreased incorporation of uridine and leucine into RNA and protein, respectively. Fludarabine is also active against non-proliferating cells.

Phase I studies in humans have demonstrated that fludarabine is rapidly converted to the active metabolite, 2-fluoro-ara-A, within minutes after intravenous infusion. Consequently, clinical pharmacology studies have focused on 2-fluoro-ara-A pharmacokinetics. After the five daily doses of 25 mg 2-fluoro-ara-AMP/m² to cancer patients infused over 30 minutes, 2-fluoro-ara-A concentrations show a moderate accumulation. During a 5-day treatment schedule, 2-fluoro-ara-A plasma trough levels increased by a factor of about 2. The terminal half-life of 2-fluoro-ara-A was estimated as approximately 20 hours. In vitro, plasma protein binding of fludarabine ranged between 19% and 29%.

The schedule and method of fludarabine administration varies with the program of therapy to be used. Fludarabine may be given orally, by intravenous infusion or injection, or subcutaneously, for example.

c. Cladribine

In some embodiments, the pyrimidine analog antimetabolite is cladribine. Cladribine is a chlorinated nucleotide analog of 2′-Deoxyadenosine that is partially resistant to deamination by adenosine deaminase. The chemical name for cladribine is 2-chloro-6-amino-9-(2-deoxy-β-D-erythropentofuranosyl) purine (2-CdaA). It has the molecular formula C₁₀H₁₂ClN₅O₃ (M.W. 285.7 g/mol). It is a chemotherapy medication used to treat hairy-cell leukemia, chronic lymphocytic leukemia, and non-Hodgkin's lymphoma, for example.

The selective toxicity of 2-chloro-2′-deoxy-β-D-adenosine towards certain normal and malignant lymphocyte and monocyte populations is based on the relative activities of deoxycytidine kinase and deoxynucleotidase. Cladribine passively crosses the cell membrane. In cells with a high ratio of deoxycytidine kinase to deoxynucleotidase, it is phosphorylated by deoxycytidine kinase to 2-chloro-2′-deoxy-β-D-adenosine monophosphate (2-CdAMP). Since 2-chloro-2′-deoxy-β-D-adenosine is resistant to deamination by adenosine deaminase and there is little deoxynucleotide deaminase in lymphocytes and monocytes, 2-CdAMP accumulates intracellularly and is subsequently converted into the active triphosphate deoxynucleotide, 2-chloro-2′-deoxy-β-D-adenosine triphosphate (2-CdATP). It is postulated that cells with high deoxycytidine kinase and low deoxynucleotidase activities will be selectively killed by 2-chloro-2′-deoxy-β-D-adenosine as toxic deoxynucleotides accumulate intracellularly.

Cells containing high concentrations of deoxynucleotides are unable to properly repair single-strand DNA breaks. The broken ends of DNA activate the enzyme poly (ADP-ribose) polymerase resulting in NAD and ATP depletion and disruption of cellular metabolism. There is evidence, also, that 2-CdATP is incorporated into the DNA of dividing cells, resulting in impairment of DNA synthesis. Thus, 2-chloro-2′-deoxy-β-D-adenosine can be distinguished from other chemotherapeutic agents affecting purine metabolism in that it is cytotoxic to both actively dividing and quiescent lymphocytes and monocytes, inhibiting both DNA synthesis and repair.

In a clinical investigation, 17 patients with hairy cell leukemia and normal renal function were treated for 7 days with the recommended treatment regimen of cladribine (0.09 mg/kg/day) by continuous intravenous infusion. The mean steady-state serum concentration was estimated to be 5.7 ng/mL with an estimated systemic clearance of 663.5 mL/h/kg when cladribine was given by continuous infusion over 7 days. In hairy cell leukemia patients, there does not appear to be a relationship between serum concentrations and ultimate clinical outcome. In another study, 8 patients with hematologic malignancies received a two (2) hour infusion of cladribine (0.12 mg/kg). The mean end-of-infusion plasma cladribine concentration was 48±19 ng/mL. For 5 of these patients, the disappearance of cladribine could be described by either a biphasic or triphasic decline. For these patients with normal renal function, the mean terminal half-life was 5.4 hours. Mean values for clearance and steady-state volume of distribution were 978±422 mL/h/kg and 4.5±2.8 L/kg, respectively.

Cladribine plasma concentration after intravenous administration declines multi-exponentially with an average half-life of 6.7+/−2.5 hours. In general, the apparent volume of distribution of cladribine is approximately 9 L/kg, indicating an extensive distribution in body tissues. Cladribine penetrates into cerebrospinal fluid. One report indicates that concentrations are approximately 25% of those in plasma.

The schedule and method of cladribine administration varies with the program of therapy to be used. Cladribine may be given orally, by intravenous infusion or injection, or subcutaneously, for example.

d. Clofarabine

In some embodiments, the pyrimidine analog antimetabolite is clofarabine. Clofarabine is a purine nucleoside analogue consisting of a 6-amino-2-chloropurin-9-yl group attached to the 1beta position of 2′-deoxy-2′-fluoro-D-arabinofuranose. The chemical name for clofarabine is 2 2-Chloro-9-(2-deoxy-2-fluoro-beta-D-arabinofuranosyl)-9H-purin-6-amine 2-Chloro-9-(2-deoxy-2-fluoro-beta-D-arabinofuranosyl)-9H-purin-6-amine. It has the molecular formula C₁₀H₁₁ClFN₅O₃ (M.W. 303.68 g/mol). It is a chemotherapy medication used to treat relapsed or refractory acute lymphocytic (lymphoblastic) leukemia, acute myeloid leukemia, myelodysplastic syndromes, and myeloid blast phase chronic myeloid leukemia (CML), for example.

Clofarabine is sequentially metabolized intracellularly to the 5′-monophosphate metabolite by deoxycytidine kinase and mono and di-phospho-kinases to the active 5′-triphosphate metabolite. Clofarabine has affinity for the activating phosphorylating enzyme, deoxycytidine kinase, equal to or greater than that of the natural substrate, deoxycytidine. Clofarabine inhibits DNA synthesis by decreasing cellular deoxynucleotide triphosphate pools through an inhibitory action on ribonucleotide reductase, and by terminating DNA chain elongation and inhibiting repair through incorporation into the DNA chain by competitive inhibition of DNA polymerases. The affinity of clofarabine triphosphate for these enzymes is similar to or greater than that of deoxyadenosine triphosphate. In preclinical models, clofarabine has demonstrated the ability to inhibit DNA repair by incorporation into the DNA chain during the repair process. Clofarabine 5′-triphosphate also disrupts the integrity of mitochondrial membrane, leading to the release of the proapoptotic mitochondrial proteins, cytochrome C and apoptosis inducing factor, leading to programmed cell death. Clofarabine is cytotoxic to rapidly proliferating and quiescent cancer cell types in vitro.

The population pharmacokinetics of clofarabine were studied in 40 pediatric patients aged 2 to 19 years (21 males/19 females) with relapsed or refractory acute lymphoblastic leukemia (ALL) or acute myelogenous leukemia (AML). At the given 52 mg/m² dose, similar concentrations were obtained over a wide range of body surface areas (BSAs). Clofarabine was 47% bound to plasma proteins, predominantly to albumin. Based on noncompartmental analysis, systemic clearance and volume of distribution at steady-state were 28.8 L/h/m² and 172 L/m², respectively. The terminal half-life was 5.2 hours. No apparent difference in pharmacokinetics was observed between patients with ALL and AML or between males and females. No relationship between clofarabine or clofarabine triphosphate exposure and toxicity or response was found in this population. Based on 24-hour urine collections in the pediatric studies, 49-60% of the dose is excreted in the urine unchanged. In vitro studies using isolated human hepatocytes indicate very limited metabolism (0.2%).

The schedule and method of clofarabine administration varies with the program of therapy to be used. Clofarabine may be given by intravenous infusion or injection, for example.

e. Other Pyrimidine Analog Antimetabolites

The analog 5-azacytidine (4-amino-1-p-D-ribofuranosyl-1,3,5-triazin-2(1/-/)-one) inhibits DNA methyltransferase, causing hypomethylation of DNA, and is also incorporated directly into both RNA and DNA resulting in cell death; 5-azacytidine is incorporated into RNA more frequently than into DNA. The analog 5-azacytidine is used in the treatment of myelodysplastic syndrome and acute myeloid leukemia, for example.

Gemcitabine is 4-amino-1-[(2R,4R,5R)-3,3-difluoro-4-hydroxy-5-(hydroxymethyl)oxolan-2-yl]pyrimidin-2-one. Gemcitabine is used for treating pancreatic and in combination with cisplatin for advanced or metastatic bladder cancer and advanced or metastatic non-small cell lung cancer, as well as in various combinations for ovarian cancer or breast cancer. Gemcitabine is hydrophilic and is transported into cells via molecular transporters for nucleosides, particularly SLC29A1 SLC28A1, and SLC28A3. Gemcitabine is then phosphorylated to gemcitabine monophosphate (dFdGMP) and then eventually to gemcitabine triphosphate. Gemcitabine then is incorporated into DNA in place of cytidine. When gemcitabine is incorporated into DNA it allows a native, or normal, nucleoside base to be added next to it. This leads to “masked chain termination” as gemcitabine is a “faulty” base, but due to its neighboring native nucleoside it eludes the cell's normal repair system (base-excision repair). Thus, incorporation of gemcitabine into the cell's DNA creates an irreparable error that leads to inhibition of further DNA synthesis, and thereby leading to cell death.

Floxuridine is 5-fluoro-1-[(2R,4S,5R)-4-hydroxy-5-(hydroxymethyl)oxolan-2-yl]pyrimidine-2,4-dione. Floxuridine is used for the treatment of colorectal cancer, kidney cancer, and stomach cancer. Floxuridine is converted in vivo to 5-fluorouracil, which interferes with both DNA and RNA synthesis; 5-fluorouracil also inhibits uracil riboside phosphorylase, which prevents the utilization of preformed uracil in RNA synthesis.

The product of metabolism of floxuridine, 5-fluorouracil, is itself used as an antimetabolite. The compound 5-fluorouracil is used to treat esophageal cancer, colon cancer, stomach cancer, pancreatic cancer, breast cancer, and cervical cancer, among others. It has the IUPAC name of 5-fluoro-1H-pyrimidine-2,4-dione and blocks the action of thymidylate synthase, inhibiting DNA synthesis.

Capecitabine is another pyrimidine antimetabolite. Capecitabine is used to treat breast cancer, gastric cancer, and colorectal cancer. Its UPAC name is pentyl N-[1-[(2R,3R,4S,5R)-3,4-dihydroxy-5-methyloxolan-2-yl]-5-fluoro-2-oxopyrimidin-4-yl]carbamate. Capecitabine is also converted to 5-fluorouracil in vivo.

Other pyrimidine antimetabolites include 6-azauracil and the additional pyridine antimetabolites disclosed in W. B. Parker, “Enzymology of Purine and Pyrimidine Antimetabolites Used in the Treatment of Cancer,” Chem. Rev. 109: 2880-2893 (2009), including the following additional agents: 6-azauracil, troxacitabine, thiarabine, and sapacitabine. 6-azauracil is 2H-1,2,4-triazine-3,5-dione. Troxacitabine is 4-amino-1-[(2S,4S)-2-(hydroxymethyl)-1,3-dioxolan-4-yl]pyrimidin-2-one. Thiarabine is 4-amino-1-[(2R,3S,4S,5R)-3,4-dihydroxy-5-(hydroxymethyl)thiolan-2-yl]pyrimidin-2-one. Sapacitabine is N-[1-[(2R,3S,4S,5R)-3-cyano-4-hydroxy-5-(hydroxymethyl)oxolan-2-yl]-2-oxopyrimidin-4-yl]hexadecanamide. Sapacitabine is a prodrug of CNDAC ((2R,3S,4S,5R)-2-(4-amino-2-oxopyrimidin-1-yl)-4-hydroxy-5-(hydroxymethyl)oxolane-3-carbonitrile).

Still other pyrimidine analog antimetabolites are known in the art, including 2′-deoxy-2′-methylidenecytidine compounds as disclosed in U.S. Pat. No. 5,776,488 to Mori et al., including 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, and 2′-C-cyano-2′-deoxy-1-β-d-arabinofuranosylcytosine.

3. BH3 Mimetics

In some embodiments, the disclosed methods further comprise administration of one or more BH3 mimetics to a subject or patient in need thereof. In some embodiments, the BH3 mimetic is ABT-199 (venetoclax), ABT-737, ABT-263 (navitoclax), WEHI-539, BXI-61, BXI-72, GX15-070 (obatoclax), S1, JY-1-106, apogossypolone, BI97C1 (sabutoclax), TW-37, MIM1, MS1, BH3I-1, UMI-77, or marinopyrrole A (maritoclax). In some embodiments, the BH3 mimetic is ABT-199 (venetoclax), or ABT-737, ABT-263 (navitoclax). In some embodiments, the BH3 mimetic is ABT-199.

Targeting apoptosis is an attractive approach in cancer therapy. Apoptosis is a major barrier to cancer that must be circumvented, and evasion of apoptosis is a hallmark of cancer, causing resistance to cancer chemotherapy. BCL-2 family proteins are critical regulators of apoptosis and function immediately upstream of mitochondria. BCL-2 family proteins possess conserved BCL-2 homology (BH) domains and are classified into anti- and pro-apoptotic members that are further subdivided into ‘multidomain’ proteins, which contain four BH domains (BH1 to BH4), and ‘BH3-only’ proteins. Among these proteins, the pro-apoptotic multidomain members BAX and BAK function as mitochondrial executioners and directly open pores in the mitochondrial outer membrane, resulting in the release of the apoptogenic factors such as cytochrome c and Smac/Diabro. Studies in mice lacking both Bax and Bak showed that Bax and Bak are essential inducers of mitochondrion-mediated apoptosis in response to various stimuli, including DNA damage. In contrast, anti-apoptotic multidomain members, Bcl-2, Bcl-XL and Mcl-1, inhibit the pore formation of Bax and Bak through direct binding. BH3-only proteins are critical for initiating apoptosis, functioning immediately upstream of multidomain members, and activate Bax and Bak through direct and/or indirect activation. Quadruple deficiency of Bim, Bid, Puma and Noxa abrogates apoptosis induced by various stimuli, suggesting the importance of these direct activator type BH3-only proteins in triggering Bax/Bak-mediated apoptosis induction.

In addition to their direct effect, BH3-only proteins also inactivate anti-apoptotic multidomain proteins, resulting in indirect activation of Bax and Bak. Among BH3-only proteins, BIM and PUMA appear to bind to all anti-apoptotic multidomain proteins with equal affinity, whereas the other members display differential affinity. Particularly, NOXA, an inducer of tumor suppressor p53-mediated apoptosis, shows a unique feature in that it does not bind to BCL-2, BCL-XL or BCL-W but does bind to MCL-1 and A1 with high affinity. Therefore, it is possible that differences in BH3 domain structure control altered apoptosis-induction pathways.

Thus, because the BH3-only proteins of the BCL-2 family (having only the BCL-2 homology domain BH3) can trigger apoptosis by binding to the prosurvival members of this family and neutralizing their functional activity (sequestration of the proapoptotic Bcl-2 family members), the “BH3 mimetic” concept has prompted the development of small molecules capable of mimicking BH3-only proteins and thus inducing apoptosis. The pro-apoptotic BH3 domain consists of an amphipathic α-helix and binds to the hydrophobic groove, which contains BH1, -3 and -4, of anti-apoptotic multidomain proteins, resulting in the release of sequestered pro-apoptotic proteins BAX, BAK, and the activator type B1H₃-only proteins. Released BAX and BAK activate themselves and/or are activated by released B1H₃-only proteins to induce apoptosis, suggesting that BH3 peptides or small compounds structurally similar to the BH3 domain could be utilized as therapeutic agents against cancer.

In this context, a number of natural or synthetic small molecule inhibitors of anti-apoptotic BCL-2 family proteins have been identified and analyzed for their effect against cancer cells and tumors. Among these compounds, ABT-263 (navitoclax), an orally available derivative of ABT-737 (shown to mimic B1H3-only proteins by binding to BCL-2, BCL-XL and BCL-W and effectively induce mitochondrion-mediated apoptosis in several cancer cells), has been shown to be significantly effective in most CLL patients in clinical trials, and ABT-199 (venetoclax), also has shown to be effective in patients with relapsed or refractory CLL. Other BH3 mimetics, GX15-070 (obatoclax), B1-97C1 (sabutoclax), AT-101 (gossypol) and derivatives of AT-101 have also been clinically tested. These and other BH3 mimetics are described in Nakajima and Tanaka, (2016) BH3 mimetics: Their action and efficacy in cancer chemotherapy. Integr Cancer Sci Therap. 3, incorporated by reference herein in its entirety.

4. Other Chemotherapies

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

B. Other Cancer Therapies

1. Radiotherapy

In some embodiments, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some embodiments, the radiotherapy can comprise external radiotherapy, internal radiotherapy, radioimmunotherapy, or intraoperative radiation therapy (IORT). In some embodiments, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some embodiments, the internal radiotherapy comprises interstitial brachytherapy, intracavitary brachytherapy, or intraluminal radiation therapy. In some embodiments, the radiotherapy is administered to a primary tumor.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of radiotherapy administered to a subject may be presented as a total dose of radiotherapy, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

2. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

a. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

(1) PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDIO680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

(2) CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

(3) LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

(4) TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

b. Activation of Co-Stimulatory Molecules

In some embodiments, the immunotherapy comprises an agonist of a co-stimulatory molecule. In some embodiments, the agonist comprises an activator of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

c. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

d. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

e. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in antiviral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

f. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

3. Oncolytic Virus

In some embodiments, the one or more cancer therapies comprise an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

4. Polysaccharides

In some embodiments, the one or more cancer therapies comprise polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

5. Neoantigens

In some embodiments, the one or more cancer therapies comprise neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

6. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

7. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### II. Cancer Treatment

Aspects of the present disclosure are directed to methods comprising treatment of a subject suffering from, or suspected of having, cancer. In some embodiments, the cancer is breast cancer, ovarian cancer, renal cancer, small-cell lung cancer, non-small cell lung cancer, Hodgkin's lymphoma, non-Hodgkin's lymphoma, acute myelocytic leukemia, acute lymphocytic leukemia, melanoma, gastric cancer, adrenal cancer, head and neck cancer, hepatocellular cancer, hypernephroma, bladder cancer, acute leukemias of childhood, chronic lymphocytic leukemia, prostate cancer, glioblastoma, or myeloma. In some embodiments, the cancer is an acute leukemia of childhood. In some embodiments, the cancer is acute myelocytic leukemia. In some embodiments, the cancer is lymphoma. In some embodiments, the cancer is breast cancer. In some embodiments, the lung cancer is ovarian cancer.

In some embodiments, the disclosed methods comprise treating a subject suffering from a cancer with a combination of one or more pyrimidine analog antimetabolites and an anthracene derivative. In some embodiments, the anthracene derivative is administered subsequent to the one or more pyrimidine analog antimetabolites. As disclosed herein, the sequence in which cytotoxic agents are administered surprisingly and unexpectedly affects the efficacy of the agents. Administering the anthracene derivative subsequent to the one or more pyrimidine analog antimetabolites was surprisingly found to result in a significant, synergistic, cytotoxic effect. Conversely, administering the anthracene derivative prior to the one or more pyrimidine analog antimetabolites was surprisingly found to result in an additive effect, at best, and in some cases, an antagonistic effect. Accordingly, in some embodiments, disclosed is a method for treating a subject for cancer comprising: (a) administering to the subject a therapeutically effective amount of one or more cancer therapies; and (b), subsequent to (a), administering to the subject a therapeutically effective amount of an anthracene derivative. In some embodiments, the one or more cancer therapies comprise one or more pyrimidine analog antimetabolites. Thus, in some embodiments, disclosed is a method for treating a subject for cancer comprising: (a) administering to the subject a therapeutically effective amount of one or more pyrimidine analog antimetabolites; and (b), subsequent to (a), administering to the subject a therapeutically effective amount of an anthracene derivative.

In some embodiments, the anthracene derivative is bisantrene or a derivative or analog thereof.

In some embodiments, the one or more pyrimidine analog antimetabolites comprise two or more pyrimidine antimetabolites. In some embodiments, the one or more pyrimidine analog antimetabolites comprise cytarabine, fludarabine, cladribine, clofarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, or 2′-C-cyano-2′-deoxy-arabinofuranosylcytosine, or a combination thereof. In some embodiments, the one or more pyrimidine analog antimetabolites comprise cytarabine, fludarabine, cladribine, clofarabine, or a combination thereof. In some embodiments, the one or more one or more pyrimidine analog antimetabolites comprise cytarabine. In some embodiments, the one or more one or more pyrimidine analog antimetabolites comprise fludarabine. In some embodiments, the one or more one or more pyrimidine analog antimetabolites comprise cladribine. In some embodiments, the one or more one or more pyrimidine analog antimetabolites comprise clofarabine.

In some aspects, the method further comprises administering to the subject a BH3 mimetic. In some embodiments, the BH3 mimetic is ABT-199 (venetoclax), ABT-737, ABT-263 (navitoclax), WEHI-539, BXI-61, BXI-72, GX15-070 (obatoclax), S1, JY-1-106, apogossypolone, BI97C1 (sabutoclax), TW-37, MIM1, MS1, BH3I-1, UMI-77, or marinopyrrole A (maritoclax). In some embodiments, the BH3 mimetic is ABT-199 (venetoclax), ABT-737, or ABT-263 (navitoclax). In some embodiments, the BH3 mimetic is ABT-199.

In further embodiments, disclosed is a method for improving the efficacy of one or more cancer therapies comprising administering to a subject a therapeutically effective amount of an anthracene derivative after administration of the one or more cancer therapies. In some embodiments, the one or more cancer therapies comprise one or more pyrimidine analog antimetabolites. In certain embodiments, the subject is being or has previously been treated with a cytotoxic agent(s), for example, an anthracene derivative and/or one or more pyrimidine analog antimetabolites. In some embodiments, the efficacy of the cytotoxic agent(s) is improved. Improvement in the efficacy of a cytotoxic agent(s) may be identified using tests and diagnostic methods known in the art, such as determining therapeutic activity in animal models comprising a tumor using solid tumor model evaluation methods and non-solid tumor model evaluation methods or determining therapeutic activity using clonogenicity assays, for example.

In some embodiments, the disclosed methods comprise identifying one or more subjects as being candidates for treatment with a combination of one or more cytotoxic agents, based on current or former efficacy of the cytotoxic agent(s). For example, in some embodiments, disclosed is a method comprising identifying a subject having cancer as being a candidate for treatment with a combination of one or more cancer therapies (e.g. one or more pyrimidine analog antimetabolites) and an anthracene derivative by determining that the efficacy of the current or former one or more cancer therapies and/or the anthracene derivative is or was suboptimal. In some embodiments, the disclosed methods comprise determining an optimal cancer treatment for a subject for whom a current or former cytotoxic agent is or was suboptimal. In some embodiments, a subject is given multiple types of cancer therapy, for example multiple chemotherapies.

### III. Administration of Therapeutic Compositions

The therapy provided herein comprises administration of a combination of therapeutic agents, including at least one or more first cancer therapies or cytotoxic agents and one or more second cancer therapies or cytotoxic agents. In some embodiments, the one or more first cancer therapies or cytotoxic agents comprise one or more pyrimidine analog antimetabolites. In some embodiments, the one or more second cancer therapies or cytotoxic agents comprise an anthracene derivative. In some embodiments, the therapeutic agents are administered sequentially, with the first cancer therapy or cytotoxic agent administered before the second cancer therapy or cytotoxic agent.

Embodiments of the disclosure relate to compositions and methods comprising therapeutic compositions. The different therapies may be administered in one composition or in more than one composition, such as 2 compositions, 3 compositions, or 4 compositions. In some embodiments, the first and second cancer therapies are administered in a separate composition. In some embodiments, the first and second cancer therapies are in the same composition. Various combinations of the agents may be employed.

Compositions according to the present invention can be prepared according to standard techniques and may comprise water, buffered water, saline, glycine, dextrose, iso-osmotic sucrose solutions and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. The preparation of compositions that contains the cancer therapies will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety, and purity standards as required by FDA Office of Biological Standards.

The cancer therapies of the disclosure may be administered by the same route of administration or by different routes of administration. In some embodiments, the cancer therapy is administered intraarterially, intravenously, intraperitoneally, subcutaneously, intramuscularly, intratumorally, topically, orally, transdermally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the one or more second cancer therapies or cytotoxic agents are administered within 1 week, within 2 weeks, within 3 weeks, or within 1 month after administration of the one or more first cancer therapies or cytotoxic agents. In some embodiments, the one or more second cancer therapies or cytotoxic agents are administered within 1 week after administration of the one or more first cancer therapies or cytotoxic agents. In some embodiments, the one or more second cancer therapies or cytotoxic agents are administered within 1 day, within 2 days, within 3 days, within 4 days, within 5 days, or within 6 days after administration of the one or more first cancer therapies or cytotoxic agents. In some embodiments, the one or more second cancer therapies or cytotoxic agents are administered within 1 day after administration of the one or more first cancer therapies or cytotoxic agents. In some embodiments, the one or more second cancer therapies or cytotoxic agents are administered within 23 hours, within 22 hours, within 21 hours, within 20 hours, within 19 hours, within 18 hours, within 17 hours, within 16 hours, within 15 hours, within 14 hours, within 13 hours, within 12 hours, within 11 hours, within 10 hours, within 9 hours, within 8 hours, within 7 hours, within 6 hours, within 5 hours, within 4 hours, within 3 hours, within 2 hours, or within 1 hour after administration of the one or more first cancer therapies or cytotoxic agents. In some embodiments, the one or more second cancer therapies or cytotoxic agents are administered within 12 hours after administration of the one or more first cancer therapies or cytotoxic agents.

In some embodiments of the methods disclosed herein, a single dose of the one or more first and/or second cancer therapies are administered. In some embodiments of the methods disclosed herein, multiple doses of the one or more first and/or second cancer therapies are administered. In some embodiments, the method comprises administering multiple doses of the one or more first and/or second cancer therapies, and the multiple doses are administered on 1, 2, 3, 4, 5, 6, 7, 8, 9, 10, 11, 12, 13, 14, 15, 16, 17, 18, 19, 20, 21, 22, 23, 24, 25, 26, 27, or 28 consecutive days. In some embodiments, the method comprises administering multiple doses of the one or more first and/or second cancer therapies, and the multiple doses are administered on 1, 2, 3, 4, 5, 6, 7, 8, 9, 10, 11, 12, 13, 14, 15, 16, 17, 18, 19, 20, 21, 22, 23, 24, 25, 26, 27, or 28 non-consecutive days. Administration of the multiple doses on consecutive or non-consecutive days can comprise a cycle, and the cycle may be repeated once a month for one, two, three, four, five, six, seven, eight, nine, ten, eleven, or twelve consecutive or non-consecutive months, or once a year for one, two, three, four, or five consecutive or non-consecutive years.

In some embodiments, the one or more second cancer therapies are administered after administration of every dose of the multiple doses of the one or more first cancer therapies. In some embodiments, the one or more second cancer therapies are administered between doses of the multiple doses of the one or more first cancer therapies. Thus, in some embodiments, the one or more second cancer therapies are not administered after every dose of the one or more first cancer therapies.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

In some embodiments, the one or more first cancer therapies or cytotoxic agents comprise one or more pyrimidine analog antimetabolites. In some embodiments, the one or more pyrimidine analog antimetabolites comprise cytarabine. Cytarabine may be administered to a subject in a dosage of anywhere between 1 mg/m² and 1000 mg/m². Thus, in some embodiments, the cytarabine is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 598, 599, 600, 625, 650, 675, 700, 725, 750, 775, 800, 825, 850, 875, 900, 925, 950, 975, or 1000 mg/m², any value from 1 mg/m² to 1000 mg/m², or any range or value derivable therein. In some embodiments, the cytarabine is administered at a dose of between 5 mg/m² and 500 mg/m². In some embodiments, the cytarabine is administered at a dose of between 25 mg/m² and 250 mg/m². In some embodiments, the cytarabine is administered at a dose of between 50 mg/m² and 150 mg/m². Further, cytarabine dosing schedules may be for a variety of time periods, for example up to six weeks, or as determined by one of ordinary skill in the art to which this disclosure pertains. For example, cytarabine may be administered in a dose of 100 mg/m²/day by continuous IV infusion (days 1 to 7) or 100 mg/m² IV every 12 hours (days 1 to 7).

In some embodiments, the one or more pyrimidine analog antimetabolites comprise fludarabine. Fludarabine may be administered to a subject in a dosage of anywhere between 0.25 mg/m² and 250 mg/m². Thus, in some embodiments, the fludarabine is administered at a dose of at least, at most, or about 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/m², any value from 1 mg/m² to 250 mg/m², or any range or value derivable therein. In some embodiments, the fludarabine is administered at a dose of between 1.25 mg/m² and 125 mg/m². In some embodiments, the fludarabine is administered at a dose of between 2.5 mg/m² and 60 mg/m². In some embodiments, the fludarabine is administered at a dose of between 10 mg/m² and 40 mg/m². Further, fludarabine dosing schedules may be for a variety of time periods, for example up to six weeks, or as determined by one of ordinary skill in the art to which this disclosure pertains. For example, fludarabine may be administered in a dose of 25 mg/m² administered intravenously over a period of approximately 30 minutes daily for five consecutive days. Each 5 day course of treatment may commence every 28 days.

In some embodiments, the one or more pyrimidine analog antimetabolites comprise cladribine. Cladribine may be administered to a subject in a dosage of anywhere between 0.001 mg/kg and 1 mg/kg. Thus, in some embodiments, the cladribine is administered at a dose of at least, at most, or about 0.001, 0.002, 0.003, 0.004, 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.100, 0.101, 0.102, 0.103, 0.104, 0.105, 0.106, 0.107, 0.108, 0.109, 0.110, 0.111, 0.112, 0.113, 0.114, 0.115, 0.116, 0.117, 0.118, 0.119, 0.120, 0.121, 0.122, 0.123, 0.124, 0.125, 0.126, 0.127, 0.128, 0.129, 0.130, 0.131, 0.132, 0.133, 0.134, 0.135, 0.136, 0.137, 0.138, 0.139, 0.140, 0.141, 0.142, 0.143, 0.144, 0.145, 0.146, 0.147, 0.148, 0.149, 0.150, 0.151, 0.152, 0.153, 0.154, 0.155, 0.156, 0.157, 0.158, 0.159, 0.160, 0.161, 0.162, 0.163, 0.164, 0.165, 0.166, 0.167, 0.168, 0.169, 0.170, 0.171, 0.172, 0.173, 0.174, 0.175, 0.176, 0.177, 0.178, 0.179, 0.180, 0.181, 0.182, 0.183, 0.184, 0.185, 0.186, 0.187, 0.188, 0.189, 0.190, 0.191, 0.192, 0.193, 0.194, 0.195, 0.196, 0.197, 0.198, 0.199, 0.200, 0.201, 0.202, 0.203, 0.204, 0.205, 0.206, 0.207, 0.208, 0.209, 0.210, 0.211, 0.212, 0.213, 0.214, 0.215, 0.216, 0.217, 0.218, 0.219, 0.220, 0.221, 0.222, 0.223, 0.224, 0.225, 0.226, 0.227, 0.228, 0.229, 0.230, 0.231, 0.232, 0.233, 0.234, 0.235, 0.236, 0.237, 0.238, 0.239, 0.240, 0.241, 0.242, 0.243, 0.244, 0.245, 0.246, 0.247, 0.248, 0.249, 0.250, 0.251, 0.252, 0.253, 0.254, 0.255, 0.256, 0.257, 0.258, 0.259, 0.260, 0.261, 0.262, 0.263, 0.264, 0.265, 0.266, 0.267, 0.268, 0.269, 0.270, 0.271, 0.272, 0.273, 0.274, 0.275, 0.276, 0.277, 0.278, 0.279, 0.280, 0.281, 0.282, 0.283, 0.284, 0.285, 0.286, 0.287, 0.288, 0.289, 0.290, 0.291, 0.292, 0.293, 0.294, 0.295, 0.296, 0.297, 0.298, 0.299, 0.300, 0.301, 0.302, 0.303, 0.304, 0.305, 0.306, 0.307, 0.308, 0.309, 0.310, 0.311, 0.312, 0.313, 0.314, 0.315, 0.316, 0.317, 0.318, 0.319, 0.320, 0.321, 0.322, 0.323, 0.324, 0.325, 0.326, 0.327, 0.328, 0.329, 0.330, 0.331, 0.332, 0.333, 0.334, 0.335, 0.336, 0.337, 0.338, 0.339, 0.340, 0.341, 0.342, 0.343, 0.344, 0.345, 0.346, 0.347, 0.348, 0.349, 0.350, 0.351, 0.352, 0.353, 0.354, 0.355, 0.356, 0.357, 0.358, 0.359, 0.360, 0.361, 0.362, 0.363, 0.364, 0.365, 0.366, 0.367, 0.368, 0.369, 0.370, 0.371, 0.372, 0.373, 0.374, 0.375, 0.376, 0.377, 0.378, 0.379, 0.380, 0.381, 0.382, 0.383, 0.384, 0.385, 0.386, 0.387, 0.388, 0.389, 0.390, 0.391, 0.392, 0.393, 0.394, 0.395, 0.396, 0.397, 0.398, 0.399, 0.400, 0.401, 0.402, 0.403, 0.404, 0.405, 0.406, 0.407, 0.408, 0.409, 0.410, 0.411, 0.412, 0.413, 0.414, 0.415, 0.416, 0.417, 0.418, 0.419, 0.420, 0.421, 0.422, 0.423, 0.424, 0.425, 0.426, 0.427, 0.428, 0.429, 0.430, 0.431, 0.432, 0.433, 0.434, 0.435, 0.436, 0.437, 0.438, 0.439, 0.440, 0.441, 0.442, 0.443, 0.444, 0.445, 0.446, 0.447, 0.448, 0.449, 0.450, 0.451, 0.452, 0.453, 0.454, 0.455, 0.456, 0.457, 0.458, 0.459, 0.460, 0.461, 0.462, 0.463, 0.464, 0.465, 0.466, 0.467, 0.468, 0.469, 0.470, 0.471, 0.472, 0.473, 0.474, 0.475, 0.476, 0.477, 0.478, 0.479, 0.480, 0.481, 0.482, 0.483, 0.484, 0.485, 0.486, 0.487, 0.488, 0.489, 0.490, 0.491, 0.492, 0.493, 0.494, 0.495, 0.496, 0.497, 0.498, 0.499, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, or 1 mg/kg, any value from 0.001 mg/kg to 1 mg/kg, or any range or value derivable therein. In some embodiments, the cladribine is administered at a dose of between 0.005 mg/kg and 0.5 mg/kg. In some embodiments, the cladribine is administered at a dose of between 0.01 mg/kg and 0.25 mg/kg. In some embodiments, the cladribine is administered at a dose of between 0.05 mg/kg and 0.2 mg/kg. Further, cladribine dosing schedules may be for a variety of time periods, for example up to six weeks, or as determined by one of ordinary skill in the art to which this disclosure pertains. For example, cladribine may be administered as a single course given by continuous infusion for 7 consecutive days at a dose of 0.09 mg/kg/day.

In some embodiments, the one or more pyrimidine analog antimetabolites comprise clofarabine. Clofarabine may be administered to a subject in a dosage of anywhere between 0.5 mg/m² and 500 mg/m². Thus, in some embodiments, the clofarabine is administered at a dose of at least, at most, or about 0.500, 0.501, 0.502, 0.503, 0.504, 0.505, 0.506, 0.507, 0.508, 0.509, 0.510, 0.511, 0.512, 0.513, 0.514, 0.515, 0.516, 0.517, 0.518, 0.519, 0.520, 0.521, 0.522, 0.523, 0.524, 0.525, 0.526, 0.527, 0.528, 0.529, 0.530, 0.531, 0.532, 0.533, 0.534, 0.535, 0.536, 0.537, 0.538, 0.539, 0.540, 0.541, 0.542, 0.543, 0.544, 0.545, 0.546, 0.547, 0.548, 0.549, 0.550, 0.551, 0.552, 0.553, 0.554, 0.555, 0.556, 0.557, 0.558, 0.559, 0.560, 0.561, 0.562, 0.563, 0.564, 0.565, 0.566, 0.567, 0.568, 0.569, 0.570, 0.571, 0.572, 0.573, 0.574, 0.575, 0.576, 0.577, 0.578, 0.579, 0.580, 0.581, 0.582, 0.583, 0.584, 0.585, 0.586, 0.587, 0.588, 0.589, 0.590, 0.591, 0.592, 0.593, 0.594, 0.595, 0.596, 0.597, 0.598, 0.599, 0.600, 0.601, 0.602, 0.603, 0.604, 0.605, 0.606, 0.607, 0.608, 0.609, 0.610, 0.611, 0.612, 0.613, 0.614, 0.615, 0.616, 0.617, 0.618, 0.619, 0.620, 0.621, 0.622, 0.623, 0.624, 0.625, 0.626, 0.627, 0.628, 0.629, 0.630, 0.631, 0.632, 0.633, 0.634, 0.635, 0.636, 0.637, 0.638, 0.639, 0.640, 0.641, 0.642, 0.643, 0.644, 0.645, 0.646, 0.647, 0.648, 0.649, 0.650, 0.651, 0.652, 0.653, 0.654, 0.656, 0.656, 0.657, 0.658, 0.659, 0.660, 0.661, 0.662, 0.663, 0.664, 0.665, 0.666, 0.667, 0.668, 0.669, 0.670, 0.671, 0.672, 0.673, 0.674, 0.675, 0.676, 0.677, 0.678, 0.679, 0.680, 0.681, 0.682, 0.683, 0.684, 0.685, 0.686, 0.687, 0.688, 0.689, 0.690, 0.691, 0.692, 0.693, 0.694, 0.695, 0.696, 0.697, 0.698, 0.699, 0.700, 0.701, 0.702, 0.703, 0.704, 0.705, 0.706, 0.707, 0.708, 0.709, 0.710, 0.711, 0.712, 0.713, 0.714, 0.715, 0.716, 0.717, 0.718, 0.719, 0.720, 0.721, 0.722, 0.723, 0.724, 0.725, 0.726, 0.727, 0.728, 0.729, 0.730, 0.731, 0.732, 0.733, 0.734, 0.735, 0.736, 0.737, 0.738, 0.739, 0.740, 0.741, 0.742, 0.743, 0.744, 0.745, 0.746, 0.747, 0.748, 0.749, 0.750, 0.751, 0.752, 0.753, 0.754, 0.755, 0.756, 0.757, 0.758, 0.759, 0.760, 0.761, 0.762, 0.763, 0.764, 0.765, 0.766, 0.767, 0.768, 0.769, 0.770, 0.771, 0.772, 0.773, 0.774, 0.775, 0.776, 0.777, 0.778, 0.779, 0.780, 0.781, 0.782, 0.783, 0.784, 0.785, 0.786, 0.787, 0.788, 0.789, 0.790, 0.791, 0.792, 0.793, 0.794, 0.795, 0.796, 0.797, 0.798, 0.799, 0.800, 0.801, 0.802, 0.803, 0.804, 0.805, 0.806, 0.807, 0.808, 0.809, 0.810, 0.811, 0.812, 0.813, 0.814, 0.815, 0.816, 0.817, 0.818, 0.819, 0.820, 0.821, 0.822, 0.823, 0.824, 0.825, 0.826, 0.827, 0.828, 0.829, 0.830, 0.831, 0.832, 0.833, 0.834, 0.835, 0.836, 0.837, 0.838, 0.839, 0.840, 0.841, 0.842, 0.843, 0.844, 0.845, 0.846, 0.847, 0.848, 0.849, 0.850, 0.851, 0.852, 0.853, 0.854, 0.855, 0.856, 0.857, 0.858, 0.859, 0.860, 0.861, 0.862, 0.863, 0.864, 0.865, 0.866, 0.867, 0.868, 0.869, 0.870, 0.871, 0.872, 0.873, 0.874, 0.875, 0.876, 0.877, 0.878, 0.879, 0.880, 0.881, 0.882, 0.883, 0.884, 0.885, 0.886, 0.887, 0.888, 0.889, 0.890, 0.891, 0.892, 0.893, 0.894, 0.895, 0.896, 0.897, 0.898, 0.899, 0.900, 0.901, 0.902, 0.903, 0.904, 0.905, 0.906, 0.907, 0.908, 0.909, 0.910, 0.911, 0.912, 0.913, 0.914, 0.915, 0.916, 0.917, 0.918, 0.919, 0.920, 0.921, 0.922, 0.923, 0.924, 0.925, 0.926, 0.927, 0.928, 0.929, 0.930, 0.931, 0.932, 0.933, 0.934, 0.935, 0.936, 0.937, 0.938, 0.939, 0.940, 0.941, 0.942, 0.943, 0.944, 0.945, 0.946, 0.947, 0.948, 0.949, 0.950, 0.951, 0.952, 0.953, 0.954, 0.955, 0.956, 0.957, 0.958, 0.959, 0.960, 0.961, 0.962, 0.963, 0.964, 0.965, 0.966, 0.967, 0.968, 0.969, 0.970, 0.971, 0.972, 0.973, 0.974, 0.975, 0.976, 0.977, 0.978, 0.979, 0.980, 0.981, 0.982, 0.983, 0.984, 0.985, 0.986, 0.987, 0.988, 0.989, 0.990, 0.991, 0.992, 0.993, 0.994, 0.995, 0.996, 0.997, 0.998, 0.999, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg/m², any value from 0.5 mg/m² to 500 mg/m², or any range or value derivable therein. In some embodiments, the clofarabine is administered at a dose of between 1 mg/m² and 250 mg/m². In some embodiments, the clofarabine is administered at a dose of between 5 mg/m² and 100 mg/m². In some embodiments, the clofarabine is administered at a dose of between 25 mg/m² and 75 mg/m². Further, clofarabine dosing schedules may be for a variety of time periods, for example up to six weeks, or as determined by one of ordinary skill in the art to which this disclosure pertains. For example, clofarabine may be administered as a dose of 52 mg/m² as an intravenous infusion over 2 hours daily for 5 consecutive days.

In some embodiments, the one or more first cancer therapies or cytotoxic agents further comprise one or more BH3 mimetics. The one or more BH3 mimetics may be administered to a subject in a dosage of anywhere between 1 mg/kg and 1000 mg/kg. Thus, in some embodiments, the BH3 mimetics are administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 598, 599, 600, 625, 650, 675, 700, 725, 750, 775, 800, 825, 850, 875, 900, 925, 950, 975, or 1000 mg/kg, any value from 1 mg/kg to 1000 mg/kg, or any range or value derivable therein. In some embodiments, the BH3 mimetics are administered at a dose of between 5 mg/kg and 500 mg/kg. In some embodiments, the BH3 mimetics are administered at a dose of between 25 mg/kg and 250 mg/kg. In some embodiments, the BH3 mimetics are administered at a dose of between 50 mg/kg and mg/kg. Further, dosing schedules may be for a variety of time periods, for example up to six weeks, or as determined by one of ordinary skill in the art to which this disclosure pertains.

In some embodiments, the one or more second cancer therapies or cytotoxic agents comprise one or more anthracene derivatives. In some embodiments, the one or more anthracene derivatives comprise bisantrene or a derivative or analog thereof. Bisantrene or a derivative or analog thereof may be administered to a subject in a dosage of anywhere between 0.05 mg/m² and 5000 mg/m². Thus, in some embodiments, the bisantrene or derivative or analog thereof is administered at a dose of at least, at most, or about 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.100, 0.101, 0.102, 0.103, 0.104, 0.105, 0.106, 0.107, 0.108, 0.109, 0.110, 0.111, 0.112, 0.113, 0.114, 0.115, 0.116, 0.117, 0.118, 0.119, 0.120, 0.121, 0.122, 0.123, 0.124, 0.125, 0.126, 0.127, 0.128, 0.129, 0.130, 0.131, 0.132, 0.133, 0.134, 0.135, 0.136, 0.137, 0.138, 0.139, 0.140, 0.141, 0.142, 0.143, 0.144, 0.145, 0.146, 0.147, 0.148, 0.149, 0.150, 0.151, 0.152, 0.153, 0.154, 0.155, 0.156, 0.157, 0.158, 0.159, 0.160, 0.161, 0.162, 0.163, 0.164, 0.165, 0.166, 0.167, 0.168, 0.169, 0.170, 0.171, 0.172, 0.173, 0.174, 0.175, 0.176, 0.177, 0.178, 0.179, 0.180, 0.181, 0.182, 0.183, 0.184, 0.185, 0.186, 0.187, 0.188, 0.189, 0.190, 0.191, 0.192, 0.193, 0.194, 0.195, 0.196, 0.197, 0.198, 0.199, 0.200, 0.201, 0.202, 0.203, 0.204, 0.205, 0.206, 0.207, 0.208, 0.209, 0.210, 0.211, 0.212, 0.213, 0.214, 0.215, 0.216, 0.217, 0.218, 0.219, 0.220, 0.221, 0.222, 0.223, 0.224, 0.225, 0.226, 0.227, 0.228, 0.229, 0.230, 0.231, 0.232, 0.233, 0.234, 0.235, 0.236, 0.237, 0.238, 0.239, 0.240, 0.241, 0.242, 0.243, 0.244, 0.245, 0.246, 0.247, 0.248, 0.249, 0.250, 0.251, 0.252, 0.253, 0.254, 0.255, 0.256, 0.257, 0.258, 0.259, 0.260, 0.261, 0.262, 0.263, 0.264, 0.265, 0.266, 0.267, 0.268, 0.269, 0.270, 0.271, 0.272, 0.273, 0.274, 0.275, 0.276, 0.277, 0.278, 0.279, 0.280, 0.281, 0.282, 0.283, 0.284, 0.285, 0.286, 0.287, 0.288, 0.289, 0.290, 0.291, 0.292, 0.293, 0.294, 0.295, 0.296, 0.297, 0.298, 0.299, 0.300, 0.301, 0.302, 0.303, 0.304, 0.305, 0.306, 0.307, 0.308, 0.309, 0.310, 0.311, 0.312, 0.313, 0.314, 0.315, 0.316, 0.317, 0.318, 0.319, 0.320, 0.321, 0.322, 0.323, 0.324, 0.325, 0.326, 0.327, 0.328, 0.329, 0.330, 0.331, 0.332, 0.333, 0.334, 0.335, 0.336, 0.337, 0.338, 0.339, 0.340, 0.341, 0.342, 0.343, 0.344, 0.345, 0.346, 0.347, 0.348, 0.349, 0.350, 0.351, 0.352, 0.353, 0.354, 0.355, 0.356, 0.357, 0.358, 0.359, 0.360, 0.361, 0.362, 0.363, 0.364, 0.365, 0.366, 0.367, 0.368, 0.369, 0.370, 0.371, 0.372, 0.373, 0.374, 0.375, 0.376, 0.377, 0.378, 0.379, 0.380, 0.381, 0.382, 0.383, 0.384, 0.385, 0.386, 0.387, 0.388, 0.389, 0.390, 0.391, 0.392, 0.393, 0.394, 0.395, 0.396, 0.397, 0.398, 0.399, 0.400, 0.401, 0.402, 0.403, 0.404, 0.405, 0.406, 0.407, 0.408, 0.409, 0.410, 0.411, 0.412, 0.413, 0.414, 0.415, 0.416, 0.417, 0.418, 0.419, 0.420, 0.421, 0.422, 0.423, 0.424, 0.425, 0.426, 0.427, 0.428, 0.429, 0.430, 0.431, 0.432, 0.433, 0.434, 0.435, 0.436, 0.437, 0.438, 0.439, 0.440, 0.441, 0.442, 0.443, 0.444, 0.445, 0.446, 0.447, 0.448, 0.449, 0.450, 0.451, 0.452, 0.453, 0.454, 0.455, 0.456, 0.457, 0.458, 0.459, 0.460, 0.461, 0.462, 0.463, 0.464, 0.465, 0.466, 0.467, 0.468, 0.469, 0.470, 0.471, 0.472, 0.473, 0.474, 0.475, 0.476, 0.477, 0.478, 0.479, 0.480, 0.481, 0.482, 0.483, 0.484, 0.485, 0.486, 0.487, 0.488, 0.489, 0.490, 0.491, 0.492, 0.493, 0.494, 0.495, 0.496, 0.497, 0.498, 0.499, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 750, 1000, 1250, 1500, 1750, 2000, 2500, 3000, 3500, 4000, 4500, or 5000 mg/m², any value from 0.05 mg/m² to 5000 mg/m², or any range or value derivable therein. In some embodiments, the bisantrene or a derivative or analog thereof is administered at a dose of between 0.1 mg/m² and 2500 mg/m². In some embodiments, the bisantrene or a derivative or analog thereof is administered at a dose of between 1 mg/m² and 1000 mg/m². In some embodiments, the bisantrene or a derivative or analog thereof is administered at a dose of between 50 mg/m² and 500 mg/m². Further, dosing schedules may be for a variety of time periods, for example up to six weeks, or as determined by one of ordinary skill in the art to which this disclosure pertains.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### Examples

The following examples are included to demonstrate embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### 1. The (Bis+Ara-C+ABT199) Combination Exerts Synergistic Cytotoxicity Towards AML Cell Lines

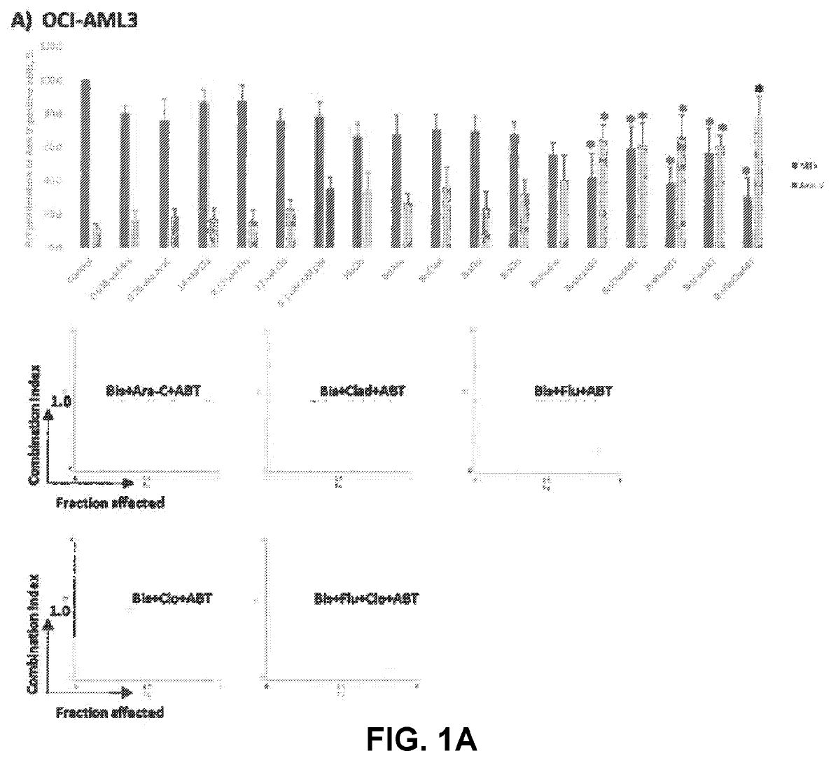

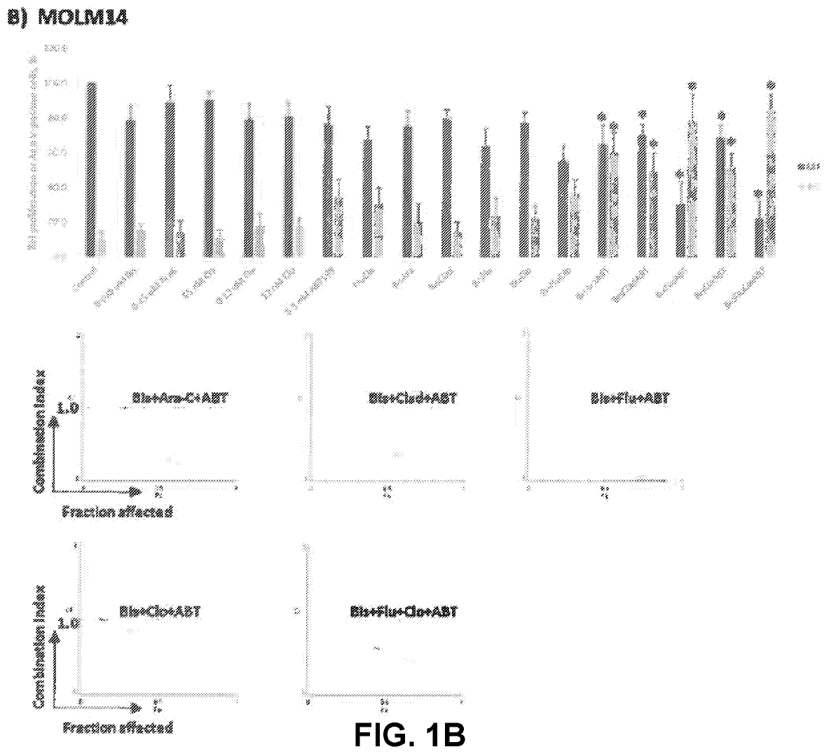

Cytarabine (Ara-C) is a common component of standard of care regimens for AML (Boddu et al, 2017). The inventors determined its cytotoxicity in OCI-AML3 and MOLM14 cell lines, alone or in combination with Bis and ABT199. Continuous exposure of OCI-AML3 cells for 48 hrs to 0.038 μM Bis, 0.26 μM Ara-C or 0.3 μM ABT199 resulted in 80%, 76% and 79% cell proliferation, respectively; combination of Bis and Ara-C resulted in 68% cell proliferation, versus control cells, and addition of ABT199 to this two-drug combination significantly reduced cell proliferation to 42% (FIG. 1A). Similar results were observed in another AML cell line. Exposure of FLT3-ITD-positive MOLM14 cells to 0.038 μM Bis, 0.45 uM Ara-C or 6.3 nM ABT199 resulted in 79%, 89% and 77% cell proliferation, respectively. The rate of proliferation decreased to 75% and 65% when cells were exposed to [Bis+Ara-C] and [Bis+Ara-C+ABT 199], respectively (FIG. 1B).

The observed cytotoxicity of [Bis+Ara-C+ABT 199] combination is consistent with the increase in Annexin V-positive cells from 27% (Bis+Ara-C) to 65% [Bis+Ara-C+ABT 199] in OCI-AML3 cells (FIG. 1A) and from 21% [Bis+Ara-C] to 60% [Bis+Ara-C+ABT 199] in MOLM14 cells (FIG. 1B), suggesting significant activation of apoptosis.

To quantitatively determine drug synergism, cells were exposed to different concentrations of individual drugs or to the three-drug combination at a constant concentration ratio and the MTT assay was performed after 48 h. Combination index (C₁) values at increasing drug effects were graphically analyzed according to the Chou-Talalay method as shown in FIG. 1 (below bar graphs). At 50% cell proliferation, or 0.5 Fa, the calculated Cl values for [Bis+Ara-C+ABT199] were 0.4 and 0.5 in OCI-AML3 and MOLM14 cell lines, respectively, suggesting strong synergism (CI<1) of the three drugs.

### 2. The Effects of Bis, Clad, and ABT199 are Synergistic in AML Cells

Cladribine (Clad), an adenosine analog, is effective for treatment of refractory AML (Zhou et al, 2019). The inventors sought to determine if Clad, like Ara-C, would provide synergistic cytotoxicity with Bis and ABT199 in AML cells. FIG. 1A shows that exposure of OCI-AML3 cells to 14 nM Clad resulted in 87% cell proliferation. When combined with 0.038 μM Bis, cell proliferation decreased to 70%; further addition of 0.3 μM ABT199 resulted in 59% proliferation. Exposure of MOLM14 cells to [Bis+Clad] or [Bis+Clad+ABT199] resulted in 80% and 70% proliferation, respectively. Analysis of OCI-AML3 cells exposed to [Bis+Clad] or [Bis+Clad+ABT199] shows 36% and 62% Ann V-positivity (FIG. 1B). In MOLM14 cells, [Bis+Clad] or [Bis+Clad+ABT199] exposure resulted in 15% and 49% Ann V-positive cells (FIG. 1B). Again, the synergism of Bis, Clad and ABT199 is suggested by CI values much less than 1 in both OCI-AML3 and MOLM14 cells.

### 3. Bis, Flu, and ABT199 Provide Synergistic Cytotoxicity in AML Cells

Fludarabine, like Ara-C and Clad, is a nucleoside analog which is indicated for treatment of leukemia patients. It is effective both for induction of remission and, because of its immunosuppressive properties (Terenzi et al., 1996), as part of pretransplant conditioning regimen for AML (Russell et al. 2002; de Lima et al., 2004: Andersson et al., 2017; Short et al., 2018). To determine its cytotoxicity with Bis in the absence or presence of ABT199, OCI-AML3 and MOLM14 cells were exposed to individual drugs or in combination. FIG. 1A shows that exposure to individual drugs resulted in 79-88% and 77-79% proliferation of OCI-AML3 and MOLM14 cells, respectively; exposure to [Bis+Flu] resulted in 70% (OCI-AML3) and 64% (MOLM14) proliferation; exposure to [Bis+Flu+ABT199] significantly inhibited proliferation to 39% (OCI-AML3) and 30% (MOLM14). Analysis of activation of apoptosis shows 16%-35% Annexin V-positivity in OCI-AML3 cells exposed to Bis, Flu, or ABT199 alone, 23% when exposed to [Bis+Flu] and 67% to [Bis+Flu+ABT199]. Similar results were obtained in MOLM14 cells. Exposure to the individual drugs resulted in 15%-35% Annexin V-positivity; 24% when exposed to [Bis+Flu] and 79% to [Bis+Flu+ABT199] combination (FIG. 1B). The synergistic cytotoxicity of Bis, Flu and ABT199 is indicated by the CI values much less than 1 in OCI-AML3 (CI=0.5) and MOLM14 (CI=0.3) cells (FIG. 1B). These results suggest that addition of ABT199 to the [Bis+Flu] combination significantly increased the cytotoxicity of the two-drug combination.

### 4. Addition of Clofarabine (Clo) to Bis and ABT199 Provides Synergistic Effects Against AML Cell Lines

Among the three nucleoside analogs tested (Ara-C, Clad, Flu), the combination of Flu with [Bis+ABT199] provided the highest cytotoxicity in OCI-AML3 and MOLM14 cells (FIGS. 1A and 1B). The inventors' previous pre-clinical (Valdez et al., 2011) and clinical (Andersson et al., 2011) studies demonstrated the efficacy of [Flu+Clo]. The inventors, therefore, sought to explore the cytotoxicity of [Bis+Clo+ABT199] and [Bis+Flu+Clo+ABT199] in AML cell lines. Exposure of OCI-AML3 cells to [Bis+Clo] resulted in 68% cell proliferation and 33% Annexin V-positivity (FIG. 1A). Addition of Flu to this two-drug combination decreased the proliferation to 55% proliferation and increased Annexin V-positive cells to 40%. Addition of ABT199 to [Bis+Clo] or [Bis+Flu+Clo] significantly decreased proliferation to 57% and 31%, respectively, and increased Annexin V-positivity to 61% and 79%. Similar results were obtained in MOLM14 cells. Exposure to [Bis+Clo], [Bis+Flu+Clo], and [Bis+Flu+Clo+ABT199] resulted in 77%, 68%, and 22% cell proliferation, respectively, and 23%, 52%, and 85% Annexin V-positivity (FIG. 1B). Analysis of their interactions showed CI values of 0.4 for both [Bis+Clo+ABT199] and [Bis+Flu+Clo+ABT199] in OCI-AML3 cells and CI values of 0.8 and 0.6 for [Bis+Clo+ABT199] and [Bis+Flu+Clo+ABT199] in MOLM14 cells (FIG. 1B), suggesting increased synergistic cytotoxicity when Bis, Flu and Clo were combined ABT199.

5. The Combination of Bis and ABT199 with Nucleoside Analog(s) Activates the Apoptosis Pathway

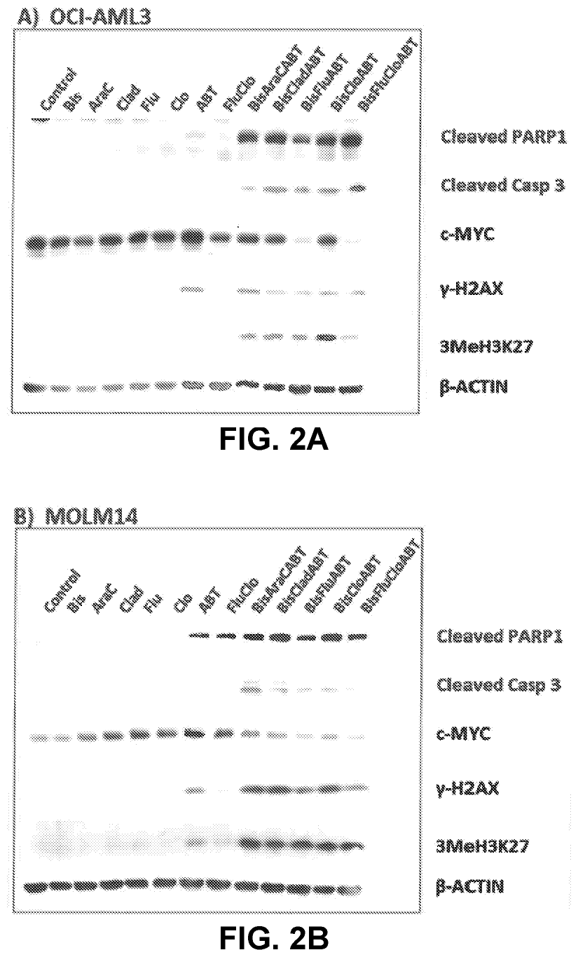

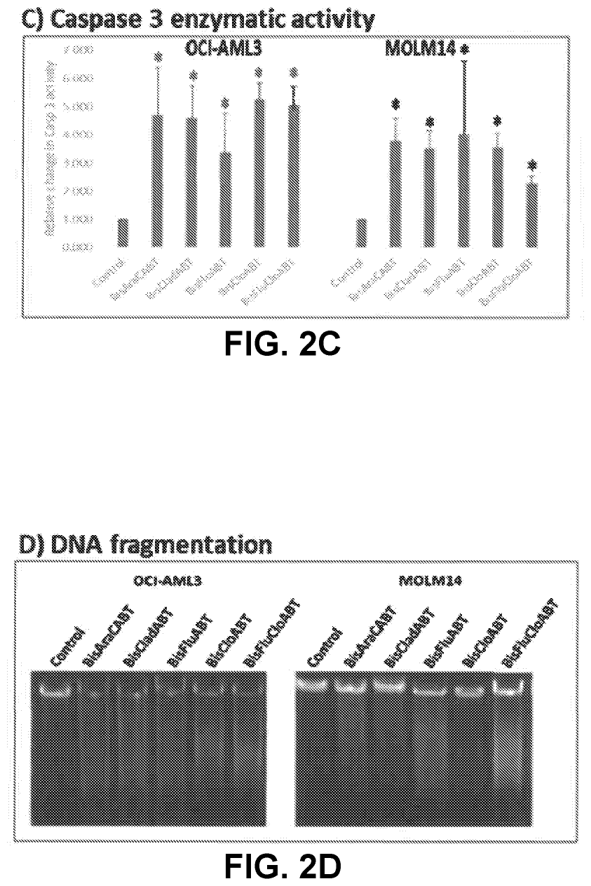

The observed increased in Ann V-positive cells suggests activation of apoptosis. The inventors, therefore, sought to determine changes in the cleavage of PARP1 and caspase 3 which are commonly used molecular markers for apoptosis activation. Exposure of OCI-AML3 cells to [Bis+nucleoside analog(s)+ABT199] resulted in the extensive cleavage of PARP1 and caspase 3 (FIG. 2A). Although exposure of MOLM14 to ABT199 or [Flu+Clo] caused cleavage of PARP1, [Bis+nucleoside analog(s)+ABT199] caused more significant cleavages of PARP1 and caspase 3 (FIG. 2B). This activation of apoptosis and the observed decrease in cell proliferation (as indicated by MTT assay) are consistent with decreased level of pro-survival c-MYC protein (FIG. 2B). The increased phosphorylation of histone 2 AX (y-H2AX) and methylation of histone 3 at Lys27 (FIGS. 2A and 2B) suggest possible activation of DNA-damage response and chromatin relaxation mediated by the three- or four-drug combinations.

To further analyze the importance of caspases in drug-mediated cell death, the inventors determined the enzymatic activity of caspase 3 in cells exposed to drug combinations. Exposure of OCI-AML3 cells to [Bis+nucleoside analog(s)+ABT 199] resulted in ˜3-5-fold increase in caspase 3 activity relative to the control cells; similar results were observed in MOLM14 cells with ˜2-4-fold increase in caspase 3 activity (FIG. 2C). Moreover, exposure of cells to these drug combinations increased DNA fragmentation, a biochemical hallmark of apoptosis (Wyllie, 1980), as determined by agarose gel analysis (FIG. 2D), suggesting activation of caspase-dependent DNase.

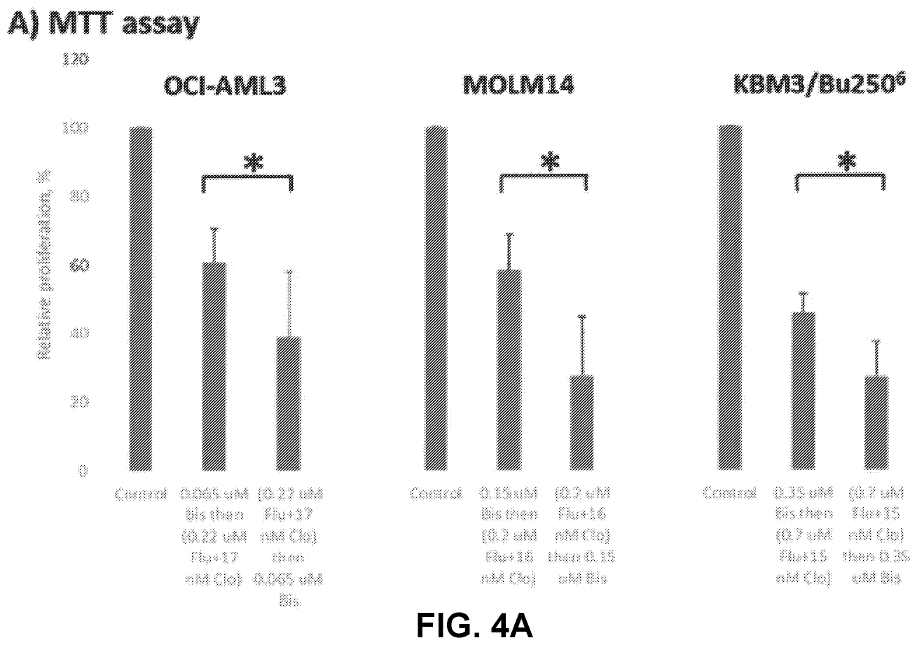

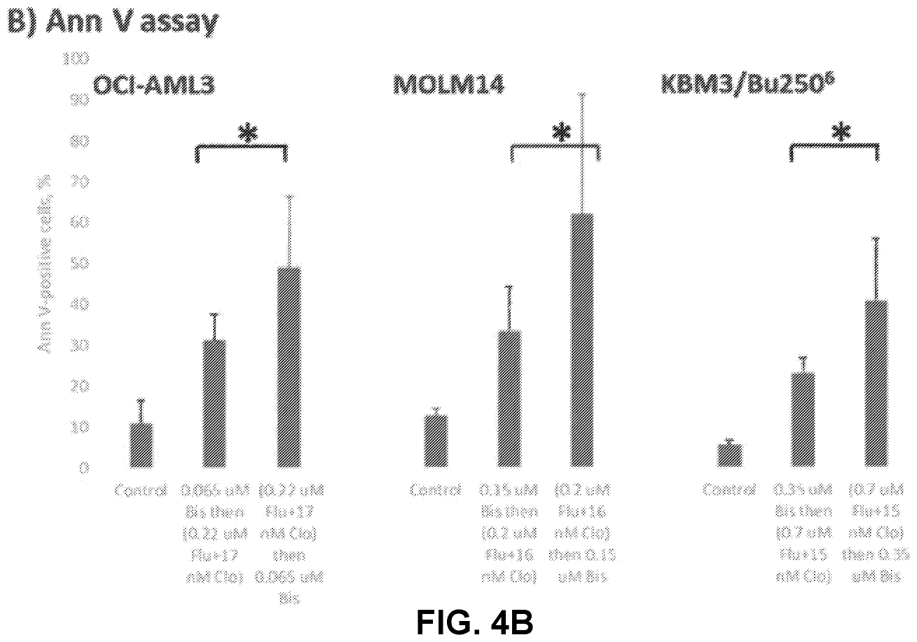

The drug-induced apoptosis might have been initiated by the effects of nucleoside analogs and Bis in the nucleus where the drugs inflicted damage to DNA, which is then communicated to the mitochondria through complex signaling pathways that decrease the levels of NAD+ and acetyl-CoA (Fang et al., 2016). The nucleoside analogs, when phosphorylated, become incorporated into DNA during synthesis, cause DNA damage and histone modifications, and induce chromatin remodeling. The relaxed chromatin probably becomes more susceptible to DNA intercalation with Bis, reminiscent of the susceptibility of relaxed chromatin to DNA alkylators as the inventors previously reported (Valdez et al., 2011). Intercalation of small molecules into DNA is known to be more efficient in less constraint chromatin (Bosire, et al., 2019). Such a process is expected to make Bis more efficient in intercalating between base pairs of the DNA and inhibiting topoisomerase II. This model is consistent with the results of the inventors' drug sequence experiment. Exposure of AML cells to [Flu+Clo] followed by Bis provided greater inhibition of proliferation compared with the reverse drug sequence (FIG. 4).

The observed fragmentation of the DNA (FIG. 2D) in cells exposed to [Bis+nucleoside analog(s)+ABT199] suggests significant drug-induced formation of DNA strand breaks. The incorporation of nucleoside analogs into the growing strand of DNA is known to collapse the DNA replication fork and inhibit DNA synthesis (Gandhi et ah, 1994), and consequently induces DNA breaks. Anthracycline-mediated poisoning of topoisomerase II also induces DNA break formation by preventing the ligation of nicked DNA strands (Marinello et al., 2018). Anthracyclines can also undergo redox reactions to generate free radicals and damage DNA (Simunek et al., 2009). All these molecular events result in activation of DNA-damage response as suggested by the phosphorylation of the ATM substrate H2AX (FIG. 2A, FIG. 2B).

Significant DNA-damage may cause cells to undergo apoptosis. Exposure of AML cells to a [Bis+nucleoside analog(s)+ABT 199] combination resulted in increased Annexin V-positivity (FIG. 1), cleavage of PARP1 and caspase 3 (FIG. 2A, FIG. 2B), caspase 3 enzymatic activity (FIG. 2C), and DNA fragmentation (FIG. 2D), suggesting activation of apoptosis.

### 6. [Bis+Nucleoside Analog(s)+ABT199] Combinations Activates the Production of ROS and Decreases Mitochondrial Membrane Potential (MMP)

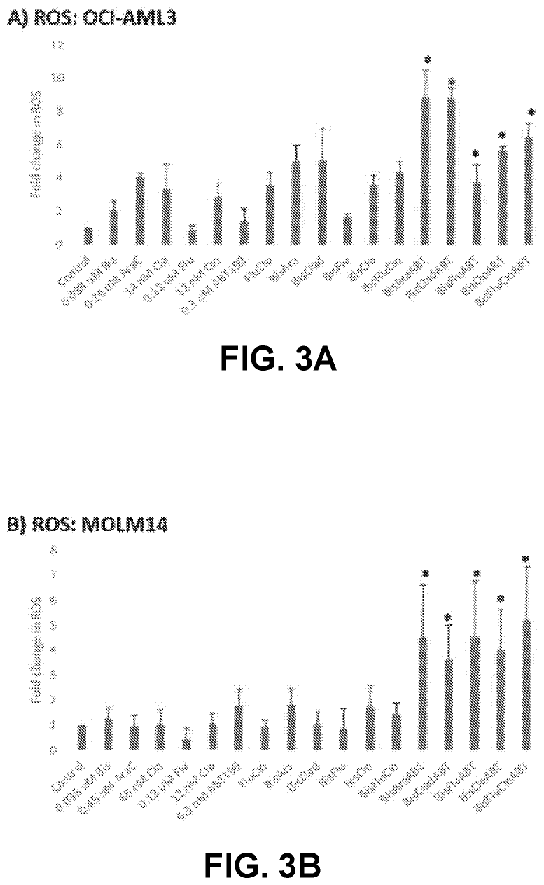

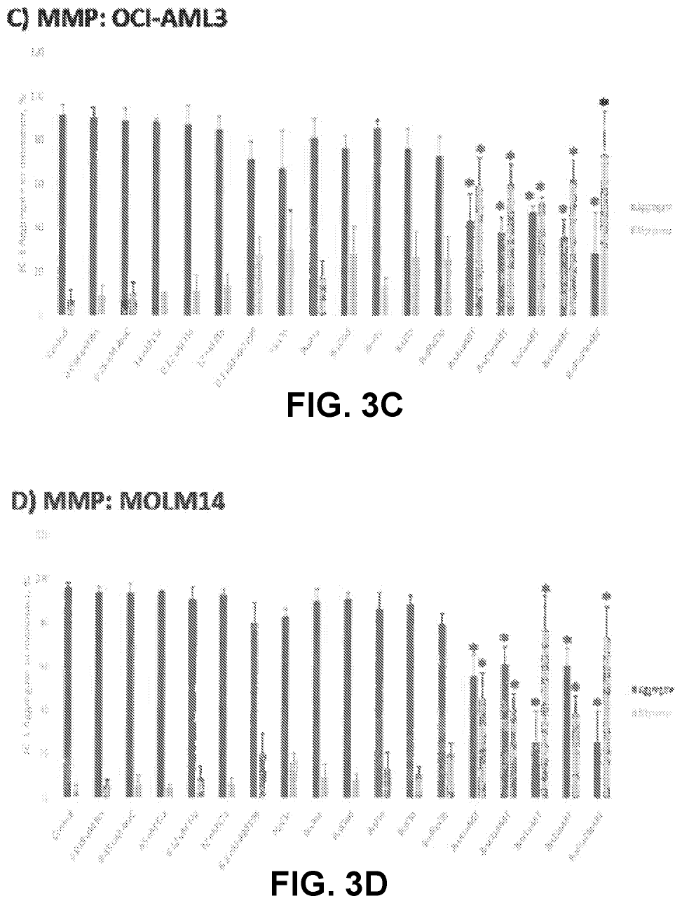

To better understand the cellular responses underlying the drug-mediated cell death, the inventors examined the production of ROS, which are known cell-death mediators. Exposure of OCI-AML3 cells to individual drags increased the production of ROS ˜1-4-fold relative to the control, while two-drug combinations increased ROS to ˜2-5-fold and three- or four-drug combinations increased ROS ˜4-9-fold (FIG. 3A). Similar results were observed in MOLM14 cells; exposure to individual or two-drug combinations resulted in ˜1-2-fold increase in ROS while exposure to [Bis+nucleoside analog(s)+ABT 199] significantly increased ROS production to ˜3.5-5-fold (FIG. 3B). The results suggest that these drug combinations have perturbed the mitochondria and increased ROS production.

To substantiate the effects of [Bis+nucleoside analog(s)+ABT199] combinations on the integrity of the mitochondria, the decrease in mitochondrial membrane potential (MMP) was determined using JC-1 reagent. The aggregated form of JC-1 in the mitochondria emits a red fluorescence and a decrease in MMP causes translocation of JC-1 reagent to the cytoplasm, where it is converted into its monomeric form that emits a green fluorescence. As shown in FIG. 3C, the control untreated OCI-AML3 cells showed 92% aggregates and 8% monomers. Exposure to individual or two-drug combinations resulted in 9%-30% JC-1 monomers whereas [Bis+nucleoside analog(s)+ABT199] exposure resulted in 52%-73% monomers, suggesting a significant leakage of JC-1 reagent from the mitochondria to the cytoplasm. Similar results were obtained in MOLM14 cells; JC-1 monomers increased from 4%-20% in cells exposed to individual or two-drug combinations to 39%-77% in cells exposed to [Bis+nucleoside analog(s)+ABT199] (FIG. 3D). Overall, these results suggest strong depolarization of the mitochondrial membrane in cells exposed to [Bis+nucleoside analog(s)+ABT199] combinations, which might have caused pro-apoptosis mitochondrial factors leakage to the cytoplasm and initiated caspase-dependent cascade of events leading to apoptosis, consistent with cleavage of caspase 3 and PARP1 (FIG. 2A, FIG. 2B).

### 7. Efficacy of Drug Sequence

The sequence of administration of drugs can be an important factor in the efficacy and interactions of 2 or more drugs (Mancini et al., 2011). For example, exposure of CG5 breast cancer cells to gemcitabine followed by doxorubicin was observed to be additive while the opposite sequence was antagonistic (Zupi et al., 2005). The inventors sought to determine whether sequence of exposure to Bis and nucleoside analogs has a differential impact on efficacy. Exposure of OCI-AML3 cells to Bis for 24 hrs followed by [Flu+Clo] for another 24 hrs resulted in ˜61% cell proliferation; the reverse sequence resulted in 39% proliferation (FIG. 4A). Similar results were obtained in another two AML cell lines; exposure of MOLM14 cells to Bis followed by [Flu+Clo] resulted in 58% cell proliferation while the reverse sequence resulted in 27% proliferation, and exposure of KBM3/Bu2506 cells to Bis followed by [Flu+Clo] resulted in 46% proliferation while the reverse sequence resulted in 27% proliferation (FIG. 4A). All differences were statistically significant. Analysis by annexin V assay provided similar significant differences. Exposure of OCI-AML3, MOLM14, and KBM3/Bu2506 cells in Bis followed by [Flu+Clo] resulted in 31%, 33%, and 23% annexin V-positive cells, respectively; the reverse sequence resulted in 49%, 62%, and 41% annexin V-positively cells, respectively (FIG. 4B). These results suggest the relevance of exposing AML cells to nucleoside analogs prior to Bis to optimize their synergistic interaction.

### 8. Exemplary Methods

Cell lines and chemicals. AML cell lines used in this study included KBM3/Bu2506, also called KBM3/Bu2506 (Valdez et al., 2008), OCI-AML3 and MOLM14 both from the laboratory of Dr. Michael Andreeff (UT MD Anderson Cancer Center, Houston, TX, USA). The KBM3/Bu2506 cell line is P53-negative and MOLM14 is positive for the presence of internal tandem repeat-FLT3 (ITD-FLT3). Cells were cultured in RPMI 1640 (Mediatech, Manassas, VA, USA) supplemented with 10% heat-inactivated fetal bovine serum (Atlanta Biologicals, Inc., Flowery Branch, GA, USA) and 100 U/ml penicillin and 100 μg/ml streptomycin (Mediatech) at 37° C. in a fully humidified atmosphere of 5% CO₂ in air. Mycoplasma contamination was determined using the EZ-PCR Mycoplasma detection kit (Biological Industries, Cromwell, CT, USA). Bisantrene-HCl (Bis) was provided by Race Oncology (Australia) through IRISYS (San Diego, CA, USA). Cytarabine, Clo and ABT199/venetoclax were obtained from SelleckChem (Houston, TX, USA). Stock solutions of Bis and Ara-C were dissolved in sterile water and phosphate-buffered saline (PBS), respectively, while Clo and ABT199 were dissolved in dimelthyl sulfoxide.

Cytotoxicity and apoptosis assays. Cells (6 ml of 0.5×10⁶ cells/ml) in T25 flasks were exposed to drugs for 48 h, aliquoted (100 μl) into 96-well plates and analyzed by the 3 (4,5 dimethylthiazol-2-yl)-2,5-diphenyl tetrazolium bromide (MTT) assay. Briefly, 30 μl of 2 mg/ml MTT reagent (Sigma-Aldrich, St. Louis, MO, USA) in PBS was added per well and incubated for 3-4 h at 37° C. The solid reaction product was dissolved by adding 100 μl of solubilization solution (0.1 N HCl in isopropanol containing 10% Triton X-100), mixing, and incubating at 37° C. for at least 1 hr. Absorbance at 570 nm was measured using a Victor X3 (Perkin Elmer Life and Analytical Sciences, Shelton, CT, USA) plate reader. The number of MTT-positive cells was determined relative to the solvent control cells.

Apoptosis was determined by flow-cytometric measurements of phosphatidylserine externalization with Annexin-V-FLUOS (Roche Diagnostics, Indianapolis, IN, USA) and a fluorescent DNA-binding marker 7-aminoactinomycin D (BD Biosciences, San Jose, CA, USA) using a Muse Cell Analyzer (EMD Millipore, Billerica, MA, USA). Drug combination effects were estimated based on the combination index (CI) values (Chao and Talalay, 1984) calculated using the CalcuSyn software (Biosoft, Ferguson, MO, USA).

Protein analysis. Western blot analysis was performed to determine mechanisms of synergism by analyzing changes in the level of key proteins and their modifications. Cells were incubated with the study drug(s) for 48 h, centrifuged, washed with ice-cold PBS and pelleted. Cells were lysed with lysis buffer (Cell Signaling Technology, Danvers, MA, USA) and total protein concentration was determined using the BCA protein Assay kit (Thermo Scientific, Rockford, IL, USA). The protein extracts were combined with the loading buffer, boiled for 5 min, and aliquots of equal amount of proteins were loaded onto polyacrylamide-SDS gels for electrophoresis. The proteins were transferred onto nitrocellulose membranes (Bio-Rad, Hercules, CA, USA). The required antibodies were added and detected using the chemiluminescent substrate Immobilon (EMD Millipore). Autoradiograms were scanned and analyzed using the UN-SCAN-IT software (Silk Scientific, Inc., Orem, UT, USA).

Analysis of mitochondrial membrane potential (MMP). The JC-1 reagent (5,5′,6,6′-tetrachloro-1,1′,3,3′-tetraethylbenzimidazolylcarbocyanine iodide) was used to determine changes in MMP using a MMP detection kit (Cayman Chemical Co., Ann Arbor, MI, USA). Cells to be analyzed were aliquoted (0.5 ml) into 5 ml tubes. Diluted (1:10 with cell growth medium, 40 μl) MMP-sensitive fluorescent dye JC-1 reagent was added to each tube, incubated at 37° C. for 20 min, and immediately analyzed by flow cytometry as described by the manufacturer. Caspase 3 assay

Cells were exposed to drugs for 48 hrs, harvested and washed with ice-cold PBS. Total cell extracts were prepared using the Caspase-3 Colorimetric Activity Assay kit (Chemicon International, Temecula, CA, USA). Total protein concentration was determined as described above. Equal amount of protein was analyzed for caspase 3 activity using the same kit.

Results are presented as the mean±standard deviation of at least three independent experiments and statistical significance of the difference between two groups was determined by Microsoft® Office Excel program, P values<0.05 were considered statistically significant.

All of the methods disclosed and claimed herein can be made and executed without undue experimentation in light of the present disclosure. While the compositions and methods of this disclosure have been described in terms of certain embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Adams J M and Cory S. The BCL-2 arbiters of apoptosis and their growing role as cancer targets. Cell Death Differ. 2018; 25:27-36.
Aldoss I, Yang D, Aribi A, et al. Efficacy of the combination of venetoclax and hypomethylating agents in relapsed/refractory acute myeloid leukemia. Haematologica. 2018; 103:e404-e407.
Andersson B S, Valdez B C, de Lima M, et al. Clofarabine±fludarabine with once daily i.v. busulfan as pretransplant conditioning therapy for advanced myeloid leukemia and MDS. Biol Blood Marrow Transplant. 2011; 17:893-900.
Andersson B S, Thall P F, Valdez B C, et al. Fludarabine with pharmacokinetically guided IV busulfan is superior to fixed-dose delivery in pretransplant conditioning of AML/MDS patients. Bone Marrow Transplant. 2017; 52:580-587.
Blum R H, Carter S K. Adriamycin: A new anticancer drug with significant clinical activity. Ann Intern Med. 1974; 80:249-259.
Boddu P C, Kantarjian H M, Ravandi F, et al. Characteristics and outcomes of older patients with secondary acute myeloid leukemia according to treatment approach. Cancer. 2017; 123:3050-60.
Bose P, Gandhi V V, Konopleva M Y. Pathways and mechanisms of venetoclax resistance. Leuk Lymphoma. 2017; 58:2026-2039.
Bosire R, Nanasi P Jr, Imre L, et al. Intercalation of small molecules into DNA in chromatin is primarily controlled by superhelical constraint. PLoS One. 2019; 14:e0224936.
Chou T C and Talalay P. Quantitative analysis of dose-effect relationships: the combined effects of multiple drugs or enzyme inhibitors. Adv Enzyme Regul. 1984; 22:27-55.
de Lima M, Couriel D, Thall P F, et al. Once-daily intravenous busulfan and fludarabine: clinical and pharmacokinetic results of a myeloablative, reduced-toxicity conditioning regimen for allogeneic stem cell transplantation in AML and MDS. Blood. 2004; 104; 857-864.
DiNardo C D, Pratz, K, Pullarkat, V, et al. Venetoclax combined with decitabine or azacitidine in treatment-naive, elderly patients with acute myeloid leukemia. Blood. 2019; 133: 7-17.
Fang E F, Scheibye-Knudsen M, Chua K F, et al. Nuclear DNA damage signaling to mitochondria in ageing. Nat Rev Mol Cell Biol. 2016; 17:308-321.
Gandhi V, Huang P, Plunkett W. Fludarabine inhibits DNA replication: a rationale for its use in the treatment of acute leukemias. Leuk Lymphoma. 1994; 14 Suppl 2:3-9.
Holmes F A, Esparza L, Yap H Y, et al. A comparative study of bisantrene given by two dose schedules in patients with metastatic breast cancer. Cancer Chemother Pharmacol. 1986; 18:157-611.
Kadia T M, Cortes J, Ravandi F, et al. Cladribine and low-dose cytarabine alternating with decitabine as front-line therapy for elderly patients with acute myeloid leukaemia: a phase 2 single-arm trial. Lancet Haematol. 2018; 5:e411-e421
Knight T, Edwards H, Taub J W, Ge Y. Evaluating venetoclax and its potential in treatment-naive acute myeloid leukemia. Cancer Manag Res. 2019; 11:3197-3213.
Mancini R, Modlin J. Chemotherapy administration sequence: A review of the literature and creation of a sequencing chart. J Hematol Oncol Pharm. 2011; 1:17-25.
Marinello J, Delcuratolo M, Capranico G. Anthracyclines as topoisomerase II poisons: From early studies to new perspectives. Int J Mol Sci. 2018; 19. pii: E3480.
Osborne C K, Von Hoff D D, Cowan J D, Sandbach J. Bisantrene, an active drug in patients with advanced breast cancer. Cancer Treat Rep. 1984; 68:357-360.
Pan R, Hogdal L J, Benito J M, et al. Selective BCL-2 inhibition by ABT-199 causes on target cell death in acute myeloid leukemia. Cancer Discov. 2014; 4: 362-375.
Popgeorgiev N, Jabbour L and Gillet G. Subcellular localization and dynamics of the Bcl-2 family of proteins. Front Cell Dev Biol. 2018; 6:13.
Qasrawi A, Bahaj W, Qasrawi L, et al. Cladribine in the remission induction of adult acute myeloid leukemia: where do we stand? Ann Hematol. 2019; 98:561-579.
Rogakou E P, Pilch D R, Orr A H, et al. DNA double-stranded breaks induce histone H2A X phosphorylation on serine 139. J Biol Chem. 1998; 273:5858-5868.
Russell J A, Tran H T, Quinlan D, et al. Once-daily intravenous busulfan given with fludarabine as conditioning for allogeneic stem cell transplantation: study of pharmacokinetics and early clinical outcomes. Biol Blood Marrow Transplant. 2002; 8:468-476.
Short N J, Kantarjian H, Ravandi F, et al. A phase I/II randomized trial of clofarabine orfludarabine added to idarubicin and cytarabine for adults with relapsed or refractory acute myeloid leukemia. Leuk Lymphoma. 2018; 59:813-820.
Simunek T, Sterba M, Popelova O, et al. Anthracycline-induced cardiotoxicity: overview of studies examining the roles of oxidative stress and free cellular iron. Pharmacol Rep. 2009; 61:154-171.
Souers A J, Leverson J D, Boghaert E R, et al. ABT-199, a potent and selective BCL-2 inhibitor, achieves antitumor activity while sparing platelets. Nat Med. 2013; 19:202-208.
Spadea A, Petti M C, Aloespiriti M A, et al. Bisantrene in relapsed and refractory acute myelogenous leukemia. Leuk Lymphoma. 1993; 9:217-20.
Terenzi A, Aristei C, Aversa F, et al. Efficacy of fludarabine as an immunosuppressor for bone marrow transplantation conditioning: preliminary results. Transplant Proc. 1996; 28:3101.
Unverferth D V, Magorien R D, Leier C V, Balcerzak S P. Doxorubicin cardiotoxicity. Cancer Treat Rev. 1982; 9:149-164.
Valdez B C, Murray D, Ramdas L, et al. Altered gene expression in busulfan-resistant human myeloid leukemia. Leuk Res. 2008; 32:1684-1697.
Valdez B C, Li Y, Murray D, Champlin R E, Andersson B S. The synergistic cytotoxicity of clofarabine, fludarabine and busulfan in AML cells involves ATM pathway activation and chromatin remodeling. Biochem Pharmacol. 2011; 81:222-232.
Valdez B C, Li Y, Murray D et al., Panobinostat and venetoclax enhance the cytotoxicity of gemcitabine, busulfan, and melphalan in multiple myeloma cells. Exp Hematol. 2020; 81:32-41.
Wenningmann N, Knapp M, Ande A, et al. Insights into doxorubicin-induced cardiotoxicity: Molecular mechanisms, preventive strategies, and early monitoring. Mol Pharmacol. 2019; 96:219-232.
Wyllie A H. Glucocorticoid induced thymocyte apoptosis is associated with endogenous endonuclease activation. Nature. 1980; 284:555-556.
Zhou A, Han Q, Song H, et al. Efficacy and toxicity of cladribine for the treatment of refractory acute myeloid leukemia: a meta-analysis. Drug Des Devel Ther. 2019; 13:1867-1878.
Zupi G, Scarsella M, D'Angelo C, et al. Potentiation of the antitumoral activity of gemcitabine and paclitaxel in combination on human breast cancer cells. Cancer Biol Ther. 2005; 4:866-871.

What is claimed:

1. A method of treating a subject for cancer, the method comprising:
(a) administering to the subject a therapeutically effective amount of one or more pyrimidine analog antimetabolites; and
(b) subsequent to (a), administering to the subject a therapeutically effective amount of an anthracene derivative.

2. A method of improving the efficacy of one or more pyrimidine analog antimetabolites comprising administering to a subject a therapeutically effective amount of an anthracene derivative after administration of the one or more pyrimidine analog antimetabolites.

3. The method of claim 1 or 2, wherein the anthracene derivative is bisantrene or a derivative or analog thereof.

4. The method of claim 3, wherein the anthracene derivative is administered at a dose of between 0.05 mg/m² to 5000 mg/m².

5. The method of claim 3 or claim 4, wherein the anthracene derivative is administered at a dose of between 0.1 mg/m² to 2500 mg/m².

6. The method of any one of claims 3-5, wherein the anthracene derivative is administered at a dose of between 1 mg/m² to 1000 mg/m².

7. The method of any one of claims 3-6, wherein the anthracene derivative is administered at a dose of between 50 mg/m² to 500 mg/m².

8. The method of any one of claims 1-7, wherein the one or more pyrimidine analog antimetabolites comprise two or more pyrimidine antimetabolites.

9. The method of any one of claims 1-8, wherein the one or more pyrimidine analog antimetabolites comprise cytarabine, fludarabine, cladribine, clofarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, 2′-C-cyano-2′-deoxy-arabinofuranosylcytosine, or a combination thereof.

10. The method of claim 8 or claim 9, wherein the one or more pyrimidine analog antimetabolites comprise cytarabine, fludarabine, cladribine, clofarabine, or a combination thereof.

11. The method of any one of claims 8-10, wherein the one or more pyrimidine analog antimetabolites comprise two or more of cytarabine, fludarabine, cladribine, and clofarabine.

12. The method of any one of claims 8-11, wherein the one or more pyrimidine analog antimetabolites comprise fludarabine and clofarabine.

13. The method of claim 10 or 11, wherein the one or more one or more pyrimidine analog antimetabolites comprise cytarabine, and wherein the cytarabine is administered at a dose of between 1 mg/m² and 1000 mg/m².

14. The method of claim 13, wherein the cytarabine is administered at a dose of between 5 mg/m² and 500 mg/m².

15. The method of claim 13 or 14, wherein the cytarabine is administered at a dose of between 25 mg/m² and 250 mg/m².

16. The method of any one of claims 13-15, wherein the cytarabine is administered at a dose of between 50 mg/m² and 150 mg/m².

17. The method of claim 10 or 11, wherein the one or more one or more pyrimidine analog antimetabolites comprise fludarabine, and wherein the fludarabine is administered at a dose of between 0.25 mg/m² and 250 mg/m².

18. The method of claim 17, wherein the fludarabine is administered at a dose of between 1.25 mg/m² and 125 mg/m².

19. The method of claim 17 or 18, wherein the fludarabine is administered at a dose of between 2.5 mg/m² and 60 mg/m².

20. The method of any one of claims 17-19, wherein the fludarabine is administered at a dose of between 10 mg/m² and 40 mg/m².

21. The method of claim 10 or 11, wherein the one or more one or more pyrimidine analog antimetabolites comprise cladribine, and wherein the cladribine is administered at a dose of between 0.001 mg/kg and 1 mg/kg.

22. The method of claim 21, wherein the cladribine is administered at a dose of between 0.005 mg/kg and 0.5 mg/kg.

23. The method of claim 21 or 22, wherein the cladribine is administered at a dose of between 0.01 mg/kg and 0.25 mg/kg.

24. The method of any one of claims 21-23, wherein the cladribine is administered at a dose of between 0.05 mg/kg and 0.2 mg/kg.

25. The method of claim 10 or 11, wherein the one or more one or more pyrimidine analog antimetabolites comprise clofarabine, and wherein the clofarabine is administered at a dose of between 0.5 mg/m² and 500 mg/m².

26. The method of claim 25, wherein the clofarabine is administered at a dose of between 1 mg/m² and 250 mg/m².

27. The method of claim 25 or 26, wherein the clofarabine is administered at a dose of between 5 mg/m² and 100 mg/m².

28. The method of any one of claims 25-27, wherein the clofarabine is administered at a dose of between 25 mg/m² and 75 mg/m².

29. The method of any one of claims 1-28, further comprising administering to the subject a BH3 mimetic.

30. The method of claim 29, wherein the BH3 mimetic is ABT-199 (venetoclax), ABT-737, ABT-263 (navitoclax), WEHI-539, BXI-61, BXI-72, GX15-070 (obatoclax), S1, JY-1-106, apogossypolone, BI97C1 (sabutoclax), TW-37, MIM1, MS1, BH3I-1, UMI-77, or marinopyrrole A (maritoclax).

31. The method of claim 29 or claim 30, wherein the BH3 mimetic is ABT-199 (venetoclax), ABT-737, or ABT-263 (navitoclax).

32. The method of any one of claims 29-31, wherein the BH3 mimetic is ABT-199.

33. The method of any one of claims 29-32, wherein the BH3 mimetic is administered at a dose of between 1 mg/kg and 1000 mg/kg.

34. The method of any one of claims 29-33, wherein the BH3 mimetic is administered at a dose of between 5 mg/kg and 500 mg/kg.

35. The method of any one of claims 29-34, wherein the BH3 mimetic is administered at a dose of between 25 mg/kg and 250 mg/kg.

36. The method of any one of claims 29-35, wherein the BH3 mimetic is administered at a dose of between 50 mg/kg and 150 mg/kg.

37. The method of any one of claims 1-36, wherein the anthracene derivative is administered within 1 week after administration of the one or more pyrimidine analog antimetabolites.

38. The method of any one of claims 1-37, wherein the anthracene derivative is administered within 1 day after administration of the one or more pyrimidine analog antimetabolites.

39. The method of any one of claims 1-38, wherein the anthracene derivative is administered within 12 hours after administration of the one or more pyrimidine analog antimetabolites.

40. The method of any one of claims 1-39, wherein multiple doses of the one or more pyrimidine analog antimetabolites are administered.

41. The method of claim 40, wherein the method comprises administering multiple doses of the one or more pyrimidine analog antimetabolites, and wherein the multiple doses are administered on 1, 2, 3, 4, 5, 6, 7, 8, 9, 10, 11, 12, 13, 14, 15, 16, 17, 18, 19, 20, 21, 22, 23, 24, 25, 26, 27, or 28 consecutive days.

42. The method of claim 40, wherein the method comprises administering multiple doses of the one or more pyrimidine analog antimetabolites, and wherein the multiple doses are administered on 1, 2, 3, 4, 5, 6, 7, 8, 9, 10, 11, 12, 13, 14, 15, 16, 17, 18, 19, 20, 21, 22, 23, 24, 25, 26, 27, or 28 non-consecutive days.

43. The method of any one of claims 40-42, wherein the anthracene derivative is administered after administration of every dose of the multiple doses of the one or more pyrimidine analog antimetabolites.

44. The method of any one of claims 40-42, wherein the anthracene derivative is administered between doses of the multiple doses of the one or more pyrimidine analog antimetabolites.

45. The method of any of claims 1-44, wherein the anthracene derivative or the one or more pyrimidine analog antimetabolites are administered intratumorally, intravenously, intramuscularly, intraperitoneally, subcutaneously, intraarticularly, intrasynovially, intrathecally, orally, topically, through inhalation, or through a combination of two or more routes of administration.

46. The method of any one of claims 1-45, wherein the anthracene derivative and the one or more pyrimidine analog antimetabolites are administered via the same route of administration.

47. The method of any one of claims 1-45, wherein the anthracene derivative and the one or more pyrimidine analog antimetabolites are administered via different routes of administration.

48. The method of any one of claims 1-47, wherein the cancer is breast cancer, ovarian cancer, renal cancer, small-cell lung cancer, non-small cell lung cancer, Hodgkin's lymphoma, non-Hodgkin's lymphoma, acute myelocytic leukemia, acute lymphocytic leukemia, melanoma, gastric cancer, adrenal cancer, head and neck cancer, hepatocellular cancer, hypernephroma, bladder cancer, acute leukemias of childhood, chronic lymphocytic leukemia, prostate cancer, glioblastoma, and myeloma.

49. The method of any one of claims 1-48, wherein the cancer is an acute leukemia of childhood.

50. The method of any one of claims 1-49, wherein the cancer is acute myelocytic leukemia.
